(19) United States
(12) Patent Application Publication
(10) Pub. No.: US 2026/0125666 A1
(43) Pub. Date: May 7, 2026

(54) METHODS AND COMPOSITIONS FOR IMPROVING CYTOSINE BASE EDITOR GENOME EDITING SPECIFICITY AND EFFICIENCY

(71) Applicant: SIGMA-ALDRICH CO. LLC, St. Louis, MO (US)

(72) Inventors: Erin Brettmann, St. Louis, MO (US); Fuqiang Chen, St. Louis, MO (US); Graeme Garvey, Defiance, MO (US)

(21) Appl. No.: 19/119,346

(22) PCT Filed: Dec. 5, 2023
(86) PCT No.: PCT/US2023/082569

§ 371 Date: Apr. 8, 2025

Related U.S. Application Data
(60) Provisional application No. 63/386,187, filed on Dec. 6, 2022.

(51) Int. Cl. C12N 9/78; C12N 9/22; C12N 15/11

(52) U.S. Cl. CPC C12N 9/78; C12N 9/226; C12N 15/11; C12Y 305/04001; C07K 2319/00; C12N 2310/20

(57) ABSTRACT

The present invention is directed toward improved materials and methods related to cytosine and adenosine base editing of genomic DNA.

### CROSS REFERENCE TO RELATED APPLICATIONS

The present application is a U.S. National Stage Application of International Application No. PCT/US2023/082569, filed Dec. 5, 2023, which claims the benefit of priority of U.S. provisional patent application No. 63/386,187, filing date Dec. 6, 2022, the entire content of each of which is incorporated herein by reference.

### SEQUENCE LISTING

The present application contains a Sequence Listing that has been submitted in XML format via Patent Center and is hereby incorporated by reference in its entirety. The XML copy, created on Dec. 6, 2023, is named P22-236-WO-PCT_SL, and is 75.3 kilobytes in size.

### BACKGROUND

Targeted genome modification is a powerful tool for genetic manipulation of DNA, including the manipulation of eukaryotic cells, embryos and animals. For example, exogenous sequences can be integrated at targeted genomic locations and/or specific endogenous DNA (e.g., chromosomal) sequences can be deleted, inactivated or modified. CRISPR-Cas9 genome editing systems have become a widespread method for introducing genetic modifications into a diverse range of cell types and organisms. While simple double-stranded DNA cleavage by Cas9 allows for gene inactivation through the formation of insertions and deletions, precision editing can be achieved through homology-directed repair (HDR) or base editing. The rate of installation of the desired edit is often quite challenging by HDR, the donor DNA can be toxic in some cell types, and the requirement for double-stranded DNA breaks (DSBs) introduces the risk of unintended deleterious repair outcomes. In contrast, base editing utilizes a deaminase domain to install a C to T or an A to G substitution at the target site without the need for a donor DNA. For cytosine base editors (CBEs), the cytosine base is deaminated to uracil, which is “read” as thymine by DNA polymerase, resulting in the installation of cytosine-to-thymine substitution. Furthermore, base editing utilizes a CRISPR effector, such as Cas9 and Cas12a, with fully or partially inactive DNA cleavage function, which prevents the formation of DSBs.

Since its first publication by Komor, et al. (Nature, 2016), cytosine base editing has been widely used in diverse organisms from bacteria to humans and is being explored for correction of pathogenic mutations in a therapeutic context. The cytosine-base editing system of Komor utilizes a catalytically dead Cas9 (dCas9) that contains Asp10Ala and His840Ala mutations that inactivate its nuclease activity while still retaining its ability to bind DNA in a guide RNA-programmed manner without cleaving the DNA backbone. As mentioned above, the deamination of cytosine is catalyzed by cytosine deaminases and results in the conversion of a cytosine to an uracil. Uracil has the base-pairing properties of thymine and, upon replication or repair, creates an A-T base pair.

However, there still exist certain drawbacks in this editing technology that need to be addressed. One of the drawbacks is the so-called “bystander editing,” wherein a neighboring cytosine residue other than the intended one within the R-loop is converted to thymine, causing an off-target effect. Several efforts to address this issue have been previously attempted with some success. These include the use of rigid peptide linkers between a deaminase and a Cas9 nickase to narrow the window of editing and thus minimize bystander editing (Tan, et al., Nat. Commun., 2019) and protein engineering on the deaminase domain to restrict the C to T editing to certain nucleotide motifs (Gehrke, et al., Nat Biotechnol, 2018; Kim, et al., Nat Biotechnol, 2017) or to reduce the frequency of editing on multiple cytosine residues within the editing window (Jin, et al., Molecular Cell, 2020). However, each of these approaches carries its own limitations.

Another issue associated with cytosine base editing is the undesired excision of the uracil intermediate during the process of C to T conversion. Uracil does not naturally occur in DNA. The most common causes of the presence of deoxyuridine in DNA are misincorporation in place of deoxythymidine and spontaneous deamination of cytosine, both of which are mutagenic. Therefore, the presence of deoxyuracil will lead to DNA damage response by uracil DNA glycosylase, resulting in excision of the uracil base. Translesion synthesis at the abasic site can lead to the installation of undesired cytosine-to-adenine or cytosine-to-guanine substitutions. Abasic sites are also prone to DNA strand breakage and, in the context of CBEs that contain Cas9 nickase activity, this can lead to double-stranded DNA breaks (DSBs), which frequently result in DNA insertions and deletions. To address these undesired repair events that might occur in cytosine base editing, the second-generation cytosine base editor (BE2) (Komor, et al., Nature, 2016) carries a phage-derived uracil glycosylase inhibitor (UGI) covalently linked to the C terminus of Cas9, which was shown to improve the rate of C to T conversion and the overall editing efficiency over the first-generation cytosine base editor (BE1) that contains no UGI (Komor, et al., Nature, 2016). An additional UGI was added to the fourth-generation cytosine base editor (BE4) to augment the effect (Komor, et al., Sci. Adv., 2017). Currently, virtually all cytosine base editors contain such composition of two UGIs tandemly linked to the C terminus of a Cas effector.

Plasmid delivery of base editor and single guide RNA (sgRNA) is currently the prevalent practice in base editing. However, plasmid delivery carries risks of increased off-target effects due to extended overexpression of the base editor, toxicity in some cell types due to sensitivity to exogenous DNA, and the potential for plasmid DNA integration into the host genome. Ribonucleoprotein (RNP) complex delivery of the base editor recombinant protein and chemically synthesized sgRNA would greatly decrease or eliminate these risks. However, expression and purification of the base editor recombinant protein from an E. coli host can be challenging, as elevated deaminase levels can be toxic to bacterial cells. To overcome this barrier, some have resorted to the use of immortalized human HEK293 cells (Jiang, et al., Sci. Adv., 2021). In addition to the protein purification challenge, the rate of editing may be lower following RNP complex delivery compared with plasmid delivery. Therefore, there is a need for base editor proteins that can be produced at commercial scale that yields high rates of cytosine-to-thymine substitutions, and that deaminate in a relatively narrow window, to allow for the installation of precision cytosine-to-thymine substitutions without bystander editing.

### SUMMARY OF THE INVENTION

Among the various aspects of the present invention are compositions and methods that substantially improve cytosine base editing specificity by using a ssDNA binding domain (SSB) to restrict bystander editing within the R-loop. Bystander editing, wherein a neighboring cytosine residue within the R-loop other than the intended cytosine is converted to thymine, poses a major limitation on cytosine base editors for use as a precision gene editing tool to correct pathogenic mutations. While not wishing to be bound by theory, we hypothesize that a SSB covalently linked to a cytosine base editor (CBE) may compete with the deaminase domain for binding to a certain portion of the single stranded DNA region formed after Cas9 target binding and R-loop formation and this would confine the deaminase activity to a smaller segment on the single stranded DNA region. SSBs are ubiquitous and vary widely in size and DNA binding footprint. For proof of concept, we fused a T4 phage derived SSB (SEQ ID NO: 1) or a T7 phage derived one (SEQ ID NO: 2) to the N terminus of a human APOBEC3A or APOBEC3B deaminase, which in turn is covalently linked to the N terminus of a SpCas9 nickase.

In some embodiments it is contemplated that the fusion protein may also contain one or more UGIs at the C terminus of the fusion protein. In other embodiments, the one or more UGIs may be free standing, i.e., not fused to the fusion protein but separate from the fusion protein. In yet other embodiments, UGI(s) fused to the fusion protein are specifically excluded from the invention. In still yet other embodiments, UGIs may be both fused and free standing. In still yet other embodiments, the present invention contemplates methods and materials for use with cytosine base editing that exclude one or more of fused and/or free standing UGIs.

To the best of our knowledge, we are the first to successfully use free standing UGI protein in cytosine base editing applications. Other attempts were by Jang, et al., (Science Advances (2021) “High purity production and precise editing of DNA base editing ribonucleoproteins” DOI: 10.1126/sciadv.abg2661) and Wang et al., (Cell Research (2017) “Enhanced base editing by co-expression of free uracil DNA glycosylase inhibitor” DOI: 10.1038/cr.2017.111). However, in these papers the UGI was overexpressed from a plasmid.

Recombinant protein was expressed and purified from E. coli and tested for C-to-T base editing in human immortalized HEK293 cells via RNP complex delivery, using chemically synthesized sgRNAs. Compared with their non SSB counterparts, all SSB-CBE fusion proteins tested improved the editing specificity by narrowing the editing window by at least one residue. The results demonstrate that this novel strategy can be utilized to reduce CBE bystander editing and thus increase the editing specificity. Compared with the prior methods that rely on deaminase mutagenesis to bias editing to certain DNA sequence motifs or rigid peptide linkers to narrow the editing window, this novel strategy may utilize SSBs with different DNA binding footprints and affinities to tailor the size of the editing window to specific needs. One of skill in the art can identify suitable SSBs for use with the present invention with the guidance of this specification (see, for example, Guo and Malik, et al., Biomolecules, 2022 12(9), 1187).

Another aspect of the present invention discloses a CBE protein fusion composition that improves editing efficiency over conventional CBEs when C-to-T editing is performed by the delivery of preassembled RNP complexes. As disclosed in the prior art, conventional CBEs contain at least one covalently linked UGI as a means to improve editing efficiency over CBEs without an UGI. This teaching is based on the delivery of a CBE-encoding plasmid DNA (Komor, et al., Nature, 2016; Komor, et al., Sci. Adv., 2017). However, we reasoned that a covalently linked UGI at the C terminus of a CBE could in fact interfere with target binding by the CRISPR effector nuclease and reduce editing efficiency in the absence of overexpression by plasmid DNA. To test this hypothesis, we constructed paired CBEs with or without a covalently linked UGI and purified the recombinant proteins from E. coli. After transfecting these proteins into human immortalized HEK293 cells in combination with chemically synthesized sgRNAs in RNP complexes, we indeed found that the CBEs without UGI were more efficient in making C-to-T conversion than the CBEs with a covalently linked UGI on all targets tested. Thus, we have uncovered a new phenomenon that contrasts with the teaching of the prior art and established an improved CBE protein fusion composition to enhance cytosine base editing by RNP complex delivery.

We further demonstrated that a free-standing recombinant UGI can be used to further improve CBE editing efficiency and specificity regardless of whether the CBE contains a covalently linked UGI. We engineered a recombinant UGI containing a Bacillus phage derived UGI, a c-MYC nuclear localization signal (NLS), and a SV40 Large T antigen NLS (SEQ ID NO: 3) and purified the protein from E. coli. Co-transfection of the recombinant UGI with a recombinant CBE substantially increased C-to-T editing efficiency. Moreover, the UGI co-transfection also improved editing product purity by reducing C-to-G/A conversion and indel formation. Although the UGI efficacy is apparent no matter whether the CBE contains a covalently linked UGI, a CBE without UGI is preferred for achieving optimal editing outcomes. It appears that the respective enhancing effects of a free-standing UGI and a CBE without a covalently linked UGI are additive to a certain degree. The composition of this novel recombinant UGI protein and its use as an effective enhancer have not been reported elsewhere to the best of our knowledge.

Another embodiment of the present invention provides a method to enhance CBE RNP editing efficiency using a sulfonated polysaccharide-based RNP electroporation enhancer. It is well recognized that RNP complex delivery of a gene editing reagent can help to reduce off-target effects and eliminate the risks of plasmid DNA-induced cell toxicity and random genome integration compared to plasmid DNA delivery. However, editing efficiency by RNP complex delivery can be lower than by plasmid DNA delivery due to rapid protein turnover in the absence of overexpression. To address this issue in base editing, we reasoned that a dextran sulfate-based SpCas9 nuclease enhancer (MilliporeSigma Product No. PEXBUF, Burlington, MA) could also be effective with CBEs. Indeed, we found that the enhancer can improve the C-to-T editing efficiency up to severalfold, particularly on difficult-to-edit target sites. Moreover, the enhancer can be used in combination with a CBE with or without a covalently linked UGI. However, it is preferred to use the enhancer in conjunction with a free standing UGI, as the enhancer may also increase other editing events, such as C-to-G/A conversion and indel formation, in the absence of a free standing UGI. This unique combination of the RNP enhancer with a recombinant UGI has not been disclosed elsewhere.

The present invention further discloses a method to extend C-to-T editing on cytosine residues that are not accessible using the conventional method. These cytosine residues are located at the 5′ end about 18 or more than 18 nucleotides upstream from a protospacer adjacent motif (PAM). In the context of the conventional 20-nt guide RNA spacer, they are either at the edge of the R-loop or in a base pairing state and are thus inaccessible to CBEs. By extending the guide RNA spacer from the conventional 18 nt to 19 nt or 20 nt to 21 nt, 22 nt, 23 nt, or more, on a synthetic sgRNA, we found that a cytosine residue located 18 nt to 23 nt from the PAM can be converted to a thymine residue by a CBE efficiently. It is conceivable that longer than 23-nt guide RNA spacers may be utilized to extend the editing range further. We also found that the spacer extension largely shifts the editing window proportionally from the PAM without markedly increasing the size of the editing window. Therefore, this strategy provides a means to extend CBE editing range on each available PAM and increases the overall genome coverage. It is contemplated that this strategy may also be applied to crRNA (CRISPR RNA) designs and tracrRNA (trans-activating crRNA). The crRNA directs a Cas effector nuclease to bind to DNA or RNA sequences complementary to the “guide” portion of the crRNA and flanked by a protospacer-adjacent motif (PAM) (for DNA targets) or a sequence without extensive complementarity to the crRNA repeat (for RNA targets).

In summary, this disclosure describes new compositions and methods for improving CBE specificity and efficiency using RNP complex delivery. Additionally, these novelties may also be utilized in the context of mRNA delivery or plasmid DNA delivery. Furthermore, they may be utilized individually or in various combinations without deviating from the principles of the present invention.

While the present invention contemplates the use of cytosine deaminase in the compositions and methods of the present invention, the present invention also contemplates the use of adenosine deaminase in the compositions and methods of the present invention. Similar to how a cytosine base editor converts a cytosine (C)—guanine (G) binding pair to a thymidine (T)—adenine (A) binding pair, an adenosine base editor converts an A-T binding pair to a G-C binding pair. Adenosine deaminases are known to one of ordinary skill in the art. See, for example, Gaudelli, et al., 2017, Nature, 551, 464-471; Richter, et al., 2020, Nature Biotechnology, 38, 883-891. One of ordinary skill in the art will be able to use adenosine deaminases in the present invention without undue experimentation in view of the teachings of this specification.

Further, while the present invention contemplates the use of T4 and/or T7 bacteriophage SSBs, the present invention also contemplates the use of other SSBs. SSBs are known to be found in all kingdoms of life (Antony & Lohman, 2019, Semin Cell Dev Biol, 86:102-111). Although SSBs have structural diversity, these structures are known to one of ordinary skill in the art (see, Antony & Lohman). Further, the physiological mechanisms by which SSBs function are also known to one of ordinary skill in the art. For example, the functional structure of SSBs is the oligonucleotide/oligosaccharide-binding (OB)-fold, a protein domain that facilitates binding to ssDNA and various protein-protein interactions. One OB-fold consists of a five-stranded β-sheet coiled to form a closed β-barrel with an α-helix capped at the end between the third and fourth β-strands. Based on the number of OB-folds, SSBs can be classified into two groups: simple SSBs which contain only one OB-fold, and higher order SSBs which contain multiple OB-folds (see, Antony & Lohman). SSBs may also or additionally comprise K homology (KH) domains, RNA recognition motifs (RRMs) and whirly domains, the structure and function of which are known to one of ordinary skill in the art (see, Guo and Malik, 2022, Biomolecules, 12:1187-1199, for a detailed description of the OB-folds and the other domains/motifs).

In one aspect, the present invention contemplates a composition suitable for modifying a cytosine residue in a DNA sequence, comprising: a single stranded DNA binding domain (SSB), a deaminase, a catalytically modified Cas protein, a guide RNA and, optionally, one or more free and/or fused uracil glycosylase inhibitors (UGI) to form a base editing RNP complex optionally with UGI. Likewise, it is contemplated that this system works with crRNA and tracrRNA-based systems.

In another aspect, the present invention contemplates that the SSB is linked directly to said deaminase.

In another aspect, the present invention contemplates that the SSB is linked indirectly to said deaminase.

In another aspect, the present invention contemplates that SSB is not covalently linked to any of the deaminase, the catalytically modified Cas protein, the guide RNA or the optional UGI.

In another aspect, the present invention contemplates that said deaminase is covalently linked to the catalytically modified Cas protein.

In another aspect, the present invention contemplates that the one or more UGI are not covalently linked to any of the deaminase, the catalytically modified Cas or the guide RNA.

In another aspect, the present invention contemplates that the one or more SSB are from viruses, prokaryotes or eukaryotes.

In another aspect, the present invention contemplates that the deaminase is selected from a cytosine deaminase or an adenosine deaminase.

In another aspect, the present invention contemplates that the catalytically modified Cas protein contains a catalytically inactive RuvC nuclease domain.

In another aspect, the present invention contemplates that the catalytically modified Cas protein contains a catalytically inactive RuvC nuclease domain and a catalytically inactive HNH nuclease domain.

In another aspect, the present invention contemplates that the catalytically modified Cas protein is a Cas9.

In another aspect, the present invention contemplates that the catalytically modified Cas protein is a Cas12.

As discussed below, other RNA guided endonucleases and their partial or completely inactivated mutants are contemplated.

In another aspect, the present invention contemplates at least one nucleic acid encoding one or more of the SSB, the deaminase, the catalytically modified Cas protein, the guide RNA and, optionally, the one or more free and/or fused UGI of the composition of the present invention.

In another aspect, the present invention contemplates that at least one expression vector comprising a nucleic acid encoding at least one or more of the SSB, the deaminase, the catalytically modified Cas protein, the guide RNA and, optionally, the one or more free and/or fused UGI of the composition of the present invention.

In another aspect, the present invention further contemplates a composition suitable for modifying a cytosine residue in a DNA sequence, comprising: a deaminase, a catalytically modified Cas protein, a guide RNA and one or more free UGI to form a base editing RNP complex with free UGI.

In another aspect, the present invention contemplates that the deaminase is covalently linked to the catalytically modified Cas protein.

In another aspect, the present invention contemplates that the one or more UGI are not covalently linked to any of the deaminase, the catalytically modified Cas protein or the guide RNA.

In another aspect, the present invention contemplates that the deaminase is selected from a cytosine deaminase or an adenosine deaminase.

In another aspect, the present invention contemplates that the catalytically modified Cas protein contains a catalytically inactive RuvC nuclease domain.

In another aspect, the present invention contemplates that the catalytically modified Cas protein contains a catalytically inactive RuvC nuclease domain and a catalytically inactive HNH nuclease domain.

In another aspect, the present invention contemplates that the catalytically modified Cas protein is a Cas9.

In another aspect, the present invention contemplates that the catalytically modified Cas protein is a Cas12.

In another aspect, the present invention contemplates that the deaminase, the catalytically modified Cas protein, the guide RNA are encoded in one or more nucleic acids and, wherein said UGI is a peptide.

In another aspect, the present invention contemplates one or more expression vectors comprising one or more of the nucleic acids of the present invention.

In another aspect, the present invention further contemplates a method for modifying a DNA sequence, said method comprising: providing, 1) an RNP complex comprising a single stranded DNA binding domain (SSB), a deaminase, a catalytically modified Cas protein and a guide RNA, and, optionally; 2) one or more free uracil glycosylase inhibitors (UGI); introducing the RNP complex and, optionally, the free UGI into a recipient cell; wherein the DNA of the recipient cell has at least one cytosine residue converted to a thymine residue.

In another aspect, the present invention contemplates a method wherein the SSB is linked directly to said deaminase.

In another aspect, the present invention contemplates a method wherein the SSB is linked indirectly to said deaminase.

In another aspect, the present invention contemplates a method wherein the deaminase is covalently linked to the catalytically modified Cas protein.

In another aspect, the present invention contemplates a method wherein the one or more UGI are not covalently linked to the deaminase, the catalytically modified Cas protein or the guide RNA.

In another aspect, the present invention contemplates a method wherein the one or more SSB are from viruses, prokaryotes or eukaryotes.

In another aspect, the present invention contemplates a method wherein the deaminase is selected from a cytosine deaminase or an adenosine deaminase.

In another aspect, the present invention contemplates a method wherein the catalytically modified Cas protein contains a catalytically inactive RuvC nuclease domain.

In another aspect, the present invention contemplates a method wherein the catalytically modified Cas protein contains a catalytically inactive RuvC nuclease domain and a catalytically inactive HNH nuclease domain.

In another aspect, the present invention contemplates a method wherein the catalytically modified Cas protein is a Cas9.

In another aspect, the present invention contemplates a method wherein the catalytically modified Cas protein is a Cas12.

In another aspect, the present invention contemplates a method wherein the RNP complex and said UGI are introduced into the recipient cell as recombinant proteins.

In another aspect, the present invention contemplates a method wherein the RNP complex and said UGI are introduced into the recipient cell as RNA.

In another aspect, the present invention contemplates a method wherein the RNP complex and said UGI are introduced into the recipient cell as at least one expression vector.

In another aspect, the present invention contemplates a method wherein the recombinant proteins or nucleic acids are introduced into the recipient cell by electroporation in the presence of a sulfonated polysaccharide-based RNP electroporation enhancer.

In another aspect, the present invention contemplates a method wherein the guide RNA has a target complementary sequence of 20 to 30 nucleotides long.

In another aspect, the present invention contemplates a method for modifying a DNA sequence, said method comprising: providing, 1) an RNP complex comprising a deaminase peptide, a catalytically modified Cas protein and a guide RNA; 2) one or more free uracil glycosylase inhibitors (UGI); introducing the RNP complex and the free UGI into a recipient cell; wherein the DNA of the recipient cell has at least one cytosine residue converted to a thymine residue.

In another aspect, the present invention contemplates a method wherein the deaminase is covalently linked to the catalytically modified Cas protein.

In another aspect, the present invention contemplates a method wherein the one or more UGI are not covalently linked to the deaminase, the catalytically modified Cas protein or the guide RNA.

In another aspect, the present invention contemplates a method wherein the deaminase is selected from a cytosine deaminase or an adenosine deaminase.

In another aspect, the present invention contemplates a method wherein the catalytically modified Cas protein contains a catalytically inactive RuvC nuclease domain.

In another aspect, the present invention contemplates a method wherein the present invention contemplates a method wherein the catalytically modified Cas protein contains a catalytically inactive RuvC nuclease domain and a catalytically inactive HNH nuclease domain.

In another aspect, the present invention contemplates a method wherein the catalytically modified Cas protein is a Cas9.

In another aspect, the present invention contemplates a method wherein the catalytically modified Cas protein is a Cas12.

In another aspect, the present invention contemplates a method wherein the RNP complex and the UGI are introduced into the recipient cell as a recombinant protein.

In another aspect, the present invention contemplates a method wherein the recombinant proteins are introduced into the recipient cell by electroporation in the presence of a sulfonated polysaccharide-based RNP electroporation enhancer.

In another aspect, the present invention contemplates a method wherein the guide RNA has a target complementary sequence of 20 to 30 nucleotides long.

In another aspect, the present invention contemplates a method for introducing a UGI into a eukaryotic cell, said method comprising: at least one recipient cell and b) at least one UGI covalently linked with at least one NLS, wherein the at least one UGI covalently linked to at least one NLS form an UGI-NLS molecule; transfecting the UGI-NLS molecule into said recipient cell.

The present invention further contemplates that the UGI-NLS molecule is a recombinant protein.

The present invention further contemplates that the UGI-NLS molecule is encoded by a nucleic acid.

The present invention contemplates a composition comprising at least one UGI covalently linked to at least one NLS to form an UGI-NLS molecule.

The present invention further contemplates a composition consisting essentially of at least one UGI covalently linked to at least one NLS to form an UGI-NLS molecule.

The present invention further contemplates that the UGI-NLS molecule is a recombinant protein.

The present invention further contemplates that the UGI-NLS molecule is encoded by a nucleic acid.

### Enumerated Embodiments

1. A composition suitable for modifying a cytosine residue in a DNA sequence, comprising: a single stranded DNA binding domain (SSB), a deaminase, a catalytically modified Cas protein, a guide RNA and, optionally, one or more free and/or fused uracil glycosylase inhibitors (UGI) to form a base editing RNP complex optionally with UGI.
2. The composition of Embodiment 1, wherein the SSB is linked directly to said deaminase.
3. The composition of Embodiment 1, wherein the SSB is linked indirectly to said deaminase.
4. The composition of Embodiments 1-3, wherein said SSB is not covalently linked to any of the deaminase, the catalytically modified Cas protein, the guide RNA or the optional UGI.
5. The composition of any of Embodiments 1-4, wherein said deaminase is covalently linked to the catalytically modified Cas protein.
6. The composition of any of Embodiments 1-5, wherein said one or more UGI are not covalently linked to any of the deaminase, the catalytically modified Cas protein or the guide RNA.
7. The composition of any of Embodiments 1-6, wherein the one or more SSB are from viruses.
8. The composition of any of Embodiments 1-6, wherein the one or more SSB are from prokaryotes.
9. The composition of any of Embodiments 1-6, wherein the one or more SSB are from eukaryotes.
10. The composition of any of Embodiments 1-9, wherein said deaminase is a cytosine deaminase.
11. The composition of any of Embodiments 1-9, wherein said an adenosine deaminase.
12. The composition of any of Embodiments 1-11, wherein the catalytically modified Cas protein contains a catalytically inactive RuvC nuclease domain.
13. The composition of any of Embodiments 1-11, wherein the catalytically modified Cas protein contains a catalytically inactive RuvC nuclease domain and a catalytically inactive HNH nuclease domain.
14. The composition of any of Embodiments 1-13, wherein the catalytically modified Cas protein is a Cas9.
15. The composition of any of Embodiments 1-13, wherein the catalytically modified Cas protein is a Cas12.
16. At least one nucleic acid encoding one or more of the SSB, the deaminase, the catalytically modified Cas protein, the guide RNA and, optionally, the one or more free and/or fused UGI of the composition of any of Embodiments 1-15.
17. At least one expression vector comprising a nucleic acid encoding at least one or more of the SSB, the deaminase, the catalytically modified Cas protein, the guide RNA and, optionally, the one or more free and/or fused UGI of the composition of any of Embodiments 1-15.
18. The composition of any one of Embodiments 1-17 further comprising a Nuclear Location Sequence (NLS).
19. A composition suitable for modifying a cytosine residue in a DNA sequence, comprising: a deaminase, a catalytically modified Cas protein, a guide RNA and one or more free UGI to form a base editing RNP complex with free UGI.
20. The composition of Embodiment 19, wherein said deaminase is covalently linked to the catalytically modified Cas protein.
21. The composition of Embodiments 19-20, wherein said one or more UGI are not covalently linked to any of the deaminase, the catalytically modified Cas protein or the guide RNA.
22. The composition of any of Embodiments 19-21, wherein said deaminase a cytosine deaminase.
23. The composition of any of Embodiments 19-21, wherein said deaminase is an adenosine deaminase
24. The composition of any of Embodiments 19-23, wherein the catalytically modified Cas protein contains a catalytically inactive RuvC nuclease domain.
25. The composition of any of Embodiments 19-23, wherein the catalytically modified Cas protein contains a catalytically inactive RuvC nuclease domain and a catalytically inactive HNH nuclease domain.
26. The composition of any of Embodiments 19-25, wherein the catalytically modified Cas protein is a Cas9.
27. The composition of any of Embodiments 19-25, wherein the catalytically modified Cas protein is a Cas12.
28. The composition of any of Embodiments 19-27, wherein said deaminase, said catalytically modified Cas protein, said guide RNA are encoded in one or more nucleic acids and, wherein said UGI is a peptide or encoded in said one or more nucleic acid.
29. One or more expression vectors comprising one or more of the nucleic acids of Embodiment 28.
30. The composition of any one of Embodiments 19-29 further comprising a Nuclear Location Sequence (NLS).
31. A method for modifying a DNA sequence, said method comprising:
   a) providing, 1) an RNP complex comprising a single stranded DNA binding domain (SSB), a deaminase, a catalytically modified Cas protein and a guide RNA and, optionally; 2) one or more free uracil glycosylase inhibitors (UGI);
   b) introducing said RNP complex and, optionally, said free UGI into a recipient cell;
   c) wherein the DNA of said recipient cell has at least one cytosine residue converted to a thymine residue.
32. The method of Embodiment 31, wherein the SSB is linked directly to said deaminase.
33. The method of Embodiment 31, wherein the SSB is linked indirectly to said deaminase.
34. The method of any of Embodiments 31-33, wherein said deaminase is covalently linked to the catalytically modified Cas protein.
35. The method of any of Embodiments 31-33, wherein said one or more UGI are not covalently linked to the deaminase, the catalytically modified Cas protein or the guide RNA.
36. The method of any of Embodiments 31-33, wherein the one or more SSB are from viruses.
37. The method of any of Embodiments 31-33, wherein the one or more SSB are from prokaryotes.
38. The method of any of Embodiments 31-33, wherein the one or more SSB are from eukaryotes.
39. The method of any of Embodiments 31-38, wherein said deaminase is a cytosine deaminase.
40. The method of any of Embodiments 31-38, wherein said deaminase is an adenosine deaminase
41. The method of any of Embodiments 31-38, wherein the catalytically modified Cas protein contains a catalytically inactive RuvC nuclease domain.
42. The method of any of Embodiments 31-38, wherein the catalytically modified Cas protein contains a catalytically inactive RuvC nuclease domain and a catalytically inactive HNH nuclease domain.
43. The method of any of Embodiments 31-40, wherein the catalytically modified Cas protein is a Cas9.
44. The method of any of Embodiments 31-40, wherein the catalytically modified Cas protein is a Cas12.
45. The method of any of Embodiments 31-44, wherein said RNP complex and said UGI are introduced into the recipient cell as recombinant proteins.
46. The method of any of Embodiments 31-44, wherein said RNP complex and said UGI are introduced into the recipient cell as RNA.
47. The method of any of Embodiments 31-44, wherein said RNP complex and said UGI are introduced into the recipient cell as at least one expression vector.
48. The method of any of Embodiments 31-45, wherein said recombinant proteins are introduced into the recipient cell by electroporation in the presence of a sulfonated polysaccharide-based RNP electroporation enhancer.
49. The method of any of Embodiments 31-48, wherein said guide RNA has a target complementary sequence of 20 to 30 nucleotides long.
50. The method of any one of Embodiments 31-49 further comprising a Nuclear Location Sequence (NLS).
51. A method for modifying a DNA sequence, said method comprising:
   a) providing, 1) an RNP complex comprising a deaminase peptide, a catalytically modified Cas protein and a guide RNA; 2) one or more free uracil glycosylase inhibitors (UGI);
   b) introducing said RNP complex and said free UGI into a recipient cell;
   c) wherein the DNA of said recipient cell has at least one cytosine residue converted to a thymine residue.
52. The method of Embodiment 51, wherein said deaminase is covalently linked to the catalytically modified Cas protein.
53. The method of Embodiment 51, wherein said one or more UGI are not covalently linked to the deaminase, the catalytically modified Cas protein or the guide RNA.
54. The method of any of Embodiments 51-53, wherein said deaminase a cytosine deaminase.
55. The method of any of Embodiments 51-53, wherein said deaminase is an adenosine deaminase
56. The method of any of Embodiments 51-52, wherein the catalytically modified Cas nuclease contains a catalytically inactive RuvC nuclease domain.
57. The method of any of Embodiments 51-52, wherein the catalytically modified Cas protein contains a catalytically inactive RuvC nuclease domain and a catalytically inactive HNH nuclease domain.
58. The method of any of Embodiments 51-57, wherein the catalytically modified Cas protein is a Cas9.
59. The method of any of Embodiments 51-57, wherein the catalytically modified Cas protein is a Cas12.
60. The method of any of Embodiments 51-53, wherein said RNP complex and said UGI are introduced into the recipient cell as a recombinant protein.
61. The method of any of Embodiments 51-53, wherein said recombinant proteins are introduced into the recipient cell by electroporation in the presence of a sulfonated polysaccharide-based RNP electroporation enhancer.
62. The method of any of Embodiments 51-53, wherein said guide RNA has a target complementary sequence of 20 to 30 nucleotides long.
63. The method of any one of Embodiments 51-62 further comprising a Nuclear Location Sequence (NLS).
64. A method for introducing a UGI into a eukaryotic cell, said method comprising:
   a) at least one recipient cell and b) at least one UGI covalently linked with at least one NLS, wherein the at least one UGI covalently linked to at least one NLS form an UGI-NLS molecule,
   b) transfecting the UGI-NLS molecule into said recipient cell.
65. The method of Embodiment 64, wherein the UGI-NLS molecule is a recombinant protein.

### BRIEF DESCRIPTION OF THE DRAWINGS

FIGS. 1A-1E show the results of CBE with an N-terminal SSB domain fusion. (A) Target: EMX1-15. (B) Target: HEKSite2. (C) Target: HBB03. (D) Target: RNF2. (E) shows a graph of C-to-T substitutions that fall within windows of given widths.

FIGS. 2A-2D show the percentage of sequencing reads containing substitutions at each cytosine residue with and without the 2×UGI fusion. (A) Target: EMX1-15. (B) Target: HEKSite2. (C) Target: HBB03. (D) Target: RNF2.

FIGS. 3A-3C show (A) the percentage of sequencing reads containing insertions or deletions plotted for the four targets; (B) the percentage of cytosine substitutions that are C-to-T; and (C) the percentage of reads containing at least one C-to-T substitution within the protospacer.

FIGS. 4A-4H show the percentage of reads with a substitution at each cytosine in the protospacer is plotted, with C-to-T (in grey), C-to-G (in white), and C-to-A (in black). A-D contain data for the minimal CBE variant, which does not contain the SSB domain: (A) target EMX1-15; (B) target HEKSite2; (C) target HBB03 (D) target RNF2. F-H contain data for the CBE variant with the N-terminal SSB domain (E) target EMX1-15; (F) target HEKSite2; (G) target HBB03 (H) target RNF2.

FIG. 5 shows results of the addition of dextran sulfate to the co-transfection mixture of each CBE variant with UGI.

FIGS. 6A-6G show the results of increasing the length of the sgRNA. A-C show the percentage of reads with a C-to-T substitution for three targets and three spacer lengths: (A) target EMX1-15; (B) target RNF2; (C) target HBB03. D-G show the percentage of reads carrying single-reside editing alleles: (D) target EMX1-15 without SSB; (E) target RNF2 without SSB; (F) target EMX1-15 with T4 SSB; (G) target RNF2 with T4 SSB.

FIGS. 7A-7E show the results of delivery of CBEs and UGI delivered as mRNA. (A) Percent GFP positive cells; (B) Percent C-to-T conversion; (C) Percent of C-to-G conversion; (D) percent C-to-A conversion; (E) Percent indels; from human K562 cells after transfection.

FIGS. 8A and 8B show the effect of using an NLS on the co-delivered UGI protein compared to a commercially available UGI that does not contain an NLS (NEB), measured as percentage of reads with any C-to-T edit. (A) target EMX1-15; (B) target HEKSite2.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention: Singleton, et al., Dictionary of Microbiology and Molecular Biology (2nd ed. 1994); The Cambridge Dictionary of Science and Technology (Walker ed., 1988); The Glossary of Genetics, 5th Ed., R. Rieger, et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

The transitional phrases “comprising,” “consisting essentially of” and “consisting of” have the meanings as given in MPEP 2111.03 (Manual of Patent Examining Procedure; United States Patent and Trademark Office, 9^{th} Ed., Revision February 2023 [R-07.2022]). Any claims using the transitional phrase “consisting essentially of” will be understood as reciting only essential elements (i.e., the basic and novel characteristics) of the invention and any other elements recited in dependent claims are understood to be non-essential to the invention recited in the claim from which they depend. Likewise, any additional elements over those claimed that are described in a prior art reference(s) are excluded from the claims by use of the transitional phrase “consisting essentially of.”

As used herein, the term “endogenous sequence” refers to a chromosomal sequence that is native to the cell.

The term “exogenous,” as used herein, refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The terms “complementary” or “complementarity” refer to the association of double-stranded nucleic acids by base pairing through specific hydrogen bonds. The base paring may be standard Watson-Crick base pairing (e.g., 5′-A G T C-3′ pairs with the complementary sequence 3′-T C A G-5′). The base pairing also may be Hoogsteen or reversed Hoogsteen hydrogen bonding. Complementarity is typically measured with respect to a duplex region and thus, excludes overhangs, for example. Complementarity between two strands of the duplex region may be partial and expressed as a percentage (e.g., 70%), if only some of the base pairs are complementary. The bases that are not complementary are “mismatched.” Complementarity may also be complete (i.e., 100%), if all the base pairs of the duplex region are complementary.

The term “homologous” refers to the extent two or more sequences are identical. Two sequences are considered to be homologous if they will hybridize to the same sequence under a defined set of conditions. Defined conditions include but are not limited to buffer formulation, temperature and sequence concentration.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “synthetic nucleic acid” refers to a nucleotide sequence synthesized in vitro (for example, in a lab and either manually or with a nucleic acid synthesizer) and in which the sequence is not found in nature. The sequence may be, for example, DNA or RNA or a modification thereof as described below, may be any length and may be any sequence of nucleotides so long as the sequence is not naturally occurring.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

The term “inactivated” in the context of the present invention may refer to the deletion of one or more amino acids or the substitution of one or more amino acids in a target protein such that one or more functions of the protein are eliminated or reduced to a level where activity is less than 75%, less than 50%, less than 40%, less than 30%, less than 20%, less than 15%, less than 10%, less than 5%, less than 4%, less than 3%, less than 2% or less than 1% of the level of activity of the active protein. In the present invention, the Cas-like protein (e.g., Cas9) is catalytically inactivated as the result of having two amino acids substituted with Alanine residues as detailed elsewhere in this specification thereby inhibiting its ability to cleave double-stranded DNA. Reduced activity may be referred to “partially inactivated.” The term “catalytically modified” in the context of the present invention refers to a subject protein that is inactivated or partially inactivated and means that one or more of the catalytic activities of the subject protein have been reduced or eliminated. The nuclease activity of the Cas protein of the present invention may be partially or fully inactivated making the Cas protein a nickase (cleaving only one DNA strand; discussed in greater detail, below) or catalytically inactive (cleaving none of the DNA strands), respectively.

The term “directly linked” with regard to proteins and polypeptides in the context of the present invention means that two proteins are joined together (i.e., fused), (e.g., via peptide bonds) to form a continuous protein or polypeptide with no added amino acid residues incorporated between the two joined/fused proteins.

The term “indirectly linked” in the context of the present invention means that one or more amino acids are incorporated between two joined/fused proteins.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### CRISPR/Cas Proteins and Systems

Understanding the present invention will be aided by understanding CRISPR/Cas protein systems in general and in the context of the present invention.

### The CRISPR/Cas9-Based System

In its basic form, the CRISPR/Cas9 system introduces a double strand break close to the binding site of a guide RNA (gRNA). A Cas9 protein and a gRNA form a ribonucleoprotein (RNP) complex. This complex itself can be transfected into a recipient cell (for example, via the use of extracellular vesicles) or a plasmid or viral vector encoding the complex can be used. The gRNA targets the complex to the desired location in the genome through formation of an R-loop—a three-stranded nucleic acid structure composed of a DNA:RNA hybrid and a displaced single-stranded DNA—where the Cas9 protein (an endonuclease) causes a double-strand DNA break where modifications to the sequence can be made. Such modifications can take the form of non-homologous end joining (NHEJ) making random small insertions or deletions (indels) or homology-directed repair (HDR). NHEJ is useful for creating knockout mutations and HDR is useful for making desired modifications to the target sequence.

### Cytosine Base Editor

As discussed above, traditional CRISPR/Cas9 technology has efficiency and specificity challenges. Cytosine base editing was developed (Komor, 2016) to increase both the efficiency and specificity of the CRISPR/Cas9 technology. Cytosine base editing allows for the direct, irreversible conversion of one target DNA base pair into another without requiring a dsDNA backbone break or a donor template. Rather, a target cytosine (C) is converted to uracil (U) by a cytidine deaminase tethered to the RNP complex. Uracil has the base-paring properties of thymine (T). Thus, upon DNA replication or repair, the targeted C:G pair is converted to a T:A pair.

In one aspect of the present invention, a ssDNA binding domain (SSB) is used to restrict bystander editing within the R-loop. Bystander editing is where a neighboring, non-target cytosine residue within the R-loop is converted to thymine. This is important since bystander editing limits the use of cytosine base editors as a precision gene editing tool (for example, to correct pathogenic mutations). The SSB is linked to the cytosine base editor (CBE) by, for example, covalent interactions. While not being limited to theory, it is believed that the SSB may compete with the CBE for binding to the ssDNA region formed after Cas9 target binding and R-loop formation.

### (I) RNA-Guided Endonucleases

RNA-guided endonucleases, such as Cas9, may comprise at least one nuclear localization signal (NLS), at least one nuclease domain, and at least one domain that interacts with a guide RNA (gRNA) to direct the endonuclease/deaminase complex of the present invention to a specific cytosine for deamination. Also known are nucleic acids encoding the RNA-guided endonucleases, as well as methods of using the RNA-guided endonucleases to modify chromosomal sequences of eukaryotic cells or embryos. The RNA-guided endonuclease interacts with specific gRNAs, each of which directs the endonuclease to a specific targeted site, at which site the deaminase converts the target cytosine to a uracil residue that can lead to the conversion of the C-G base-pair to an A-T base-pair. Since the specificity is provided by the gRNA, the RNA-based endonuclease is, essentially, universal and can be used with different gRNAs to target different genomic sequences. The methods disclosed herein can be used to target and modify specific chromosomal sequences at targeted locations in the genome of cells or embryos. Furthermore, the targeting is specific with limited off-target effects.

Many forms of guide RNAs (gRNAs) are known in the field. In general, a gRNA is an RNA molecule that can direct an RNA binding protein or endonuclease to a specific nucleic acid sequence/target site by means of base pairing. A gRNA may comprise a single RNA molecule, such as a crRNA, or two RNA molecules, such as a crRNA and a tracrRNA (or, trRNA). In some embodiments, it may additionally comprise an accessory RNA or DNA molecule. In some embodiments, a crRNA and a tracrRNA may be covalently linked together to form a chimeric guide RNA or a single guide RNA (sgRNA). It is also well established in the field that a gRNA can be introduced alone or in combination with its cognate gRNA binding protein or endonuclease into a target cell in different forms. It can be expressed from a DNA vector introduced into a target cell. It can be synthesized by in vitro transcription or by chemical reaction before being introduced into a target cell. A person of skill in the art should know that many chemical modifications are possible on a gRNA by chemical synthesis. For example, certain modifications, such as 2′-0 methyl group modification and phosphorothioate linkage modification, may be introduced into a gRNA to alter its stability or performance. Nonstandard nucleotides, such as DNA and LNA, also may be introduced into a gRNA during chemical synthesis to alter its specificity or performance. A guide RNA that may have a different form or may contain a different chemical modification may be used in conjunction with the present disclosure without deviating from the spirit of the present disclosure.

The present disclosure provides fusion proteins, wherein a fusion protein comprises a CRISPR/Cas-like protein or fragment thereof wherein the Cas-like protein is fully or partially catalytically inactivated, retaining its ability to bind DNA but without being able to generate double-strand breaks in the target DNA. Each fusion protein is guided to a specific chromosomal sequence by a specific gRNA, wherein the associated (e.g., tethered or otherwise linked) cytosine deaminase converts the target cytosine to uracil.

RNA-guided endonucleases may comprise at least one nuclear localization signal, which permits entry of the endonuclease into the nuclei of eukaryotic cells and embryos such as, for example, non-human one cell embryos. RNA-guided endonucleases also comprise at least one nuclease domain and at least one domain that interacts with a gRNA. An RNA-guided endonuclease is directed to a specific nucleic acid sequence (or target site) by a gRNA. The gRNA interacts with the RNA-guided endonuclease as well as the target site such that, once directed to the target site, the associated cytosine deaminase can convert the target cytosine to a uracil. Since the gRNA provides the specificity for the targeted cleavage the RNA-guided endonuclease is universal (providing that its ability to cleave DNA is disabled) and can be used with different gRNAs to modify different target cytosines. RNA-guided endonucleases can be proteins, can be encoded by isolated nucleic acids (i.e., RNA or DNA), can be encoded by vectors comprising nucleic acids encoding the RNA-guided endonucleases, and can be protein-RNA complexes comprising the RNA-guided endonuclease plus a gRNA.

The RNA-guided endonuclease can be derived from a clustered regularly interspersed short palindromic repeats (CRISPR)/CRISPR-associated (Cas) system. The CRISPR/Cas system can be a type I, a type II, or a type III system, as known to one of ordinary skill in the art. Non-limiting examples of suitable CRISPR/Cas proteins include Cas3, Cas4, Cas5, Cas5e (or CasD), Cas6, Cas6e, Cas6f, Cas7, Cas8al, Cas8a2, Cas8b, Cas8c, Cas9, Cas10, Cas10d, Cas12, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3,Csxl7, Csx14, Csx10, Csx16, CsaX, Csx3, Csz1, Csx15, Csf1, Csf2, Csf3, Csf4, and Cu1966. One of ordinary skill in the art will be able to modify any RNA-guided nuclease to inactivate its catalytic activity for use with a cytosine-base editor system.

In one embodiment, the RNA-guided endonuclease is derived from a type II CRISPR/Cas system. In specific embodiments, the RNA-guided endonuclease is derived from a Cas9 protein. The Cas9 protein can be from, for example, Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In general, CRISPR/Cas proteins comprise at least one RNA recognition domain and/or RNA binding domain. RNA recognition and/or RNA binding domains interact with guide RNAs. CRISPR/Cas proteins also usually comprise nuclease domains (i.e., DNase or RNase domains; but for the purposes of cytosine-base editors these are disabled), DNA binding domains, helicase domains, RNase domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity (e.g., inactivate its ability to cleave DNA), and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from a modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and an HNH-like nuclease domain. The RuvC and HNH domains work together to cut single strands to make a double-stranded break in DNA. (Jinek, et al., Science, 337: 816-821). In some embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. For example, an aspartate to alanine (D10A) conversion in a RuvC-like domain converts the Cas9-derived protein into a nickase. Likewise, a histidine to alanine (H840A or H839A) conversion in a HNH domain converts the Cas9-derived protein into a nickase. Each nuclease domain can be modified using well-known methods, such as site-directed mutagenesis, PCR-mediated mutagenesis, and total gene synthesis, as well as other methods known in the art. Modifying both of these domains results in inactivation of nuclease and nickase activity.

The RNA-guided endonuclease may comprise at least one nuclear localization signal (NLS). In general, an NLS comprises a stretch of basic amino acids. Nuclear localization signals are known in the art (see, e.g., Lange, et al., J. Biol. Chem., 2007, 282:5101-5105). For example, in one embodiment, the NLS can be a monopartite sequence, such as PKKKRKV (SEQ ID NO:30) or PKKKRRV (SEQ ID NO:31). In another embodiment, the NLS can be a bipartite sequence. In still another embodiment, the NLS can be KRPAATKKAGQAKKKK (SEQ ID NO:32). The NLS can be located at the N-terminus, the C-terminus, or in an internal location of the RNA-guided endonuclease.

In some embodiments, the RNA-guided endonuclease can further comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO:33). In another embodiment, the cell-penetrating domain can be TLM (PLSSIFSRIGDPPKKKRKV; SEQ ID NO:34), a cell-penetrating peptide sequence derived from the human hepatitis B virus. In still another embodiment, the cell-penetrating domain can be MPG (GALFLGWLGAAGSTMGAPKKKRKV; SEQ ID NO:35 or GALFLGFLGAAGSTMGAWSQPKKKRKV; SEQ ID NO:36). In an additional embodiment, the cell-penetrating domain can be Pep-1 (KETWWETWWTEWSQPKKKRKV; SEQ ID NO:37), VP22, a cell penetrating peptide from Herpes simplex virus, or a polyarginine peptide sequence. The cell-penetrating domain can be located at the N-terminus, the C-terminus, or in an internal location of the protein.

In still other embodiments, the RNA-guided endonuclease can also comprise at least one marker domain. Non-limiting examples of marker domains include fluorescent proteins, purification tags, and epitope tags. In some embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, (SEQ ID NO: 44), biotin carboxyl carrier protein (BCCP), and calmodulin.

In certain embodiments, the RNA-guided endonuclease may be part of a protein-RNA complex comprising a gRNA. The gRNA interacts with the RNA-guided endonuclease to direct the endonuclease to a specific target site, wherein, for example, the 5′ end of the guide RNA base pairs with a specific protospacer sequence.

### (II) Fusion Proteins

Another aspect of the present disclosure provides a fusion protein comprising a CRISPR/Cas-like protein or fragment thereof and, for example, one or more SSB or one or more UGI. The CRISPR/Cas-like protein is directed to a target site by a gRNA, at which site the effector modifies or effects the targeted nucleic acid sequence. In the present invention the “effector domain” is a linked deaminase. The fusion protein can further comprise at least one additional domain chosen from a nuclear localization signal, a cell-penetrating domain, or a marker domain.

### (a) CRISPR/Cas-Like Protein

The fusion protein comprises a CRISPR/Cas-like protein or a fragment thereof. CRISPR/Cas-like proteins are detailed above in section (I). The CRISPR/Cas-like protein can be located at the N-terminus, the C-terminus, or in an internal location of the fusion protein.

In some embodiments, the CRISPR/Cas-like protein of the fusion protein can be derived from a Cas9 protein. Cas9-derived proteins can be wild type, modified, or a fragment thereof. In some embodiments of the present invention, the Cas9-derived protein is modified to inactivate both functional nuclease domains (either a RuvC-like or an HNH-like nuclease domain). In some embodiments, both of the RuvC-like nuclease domain and the HNH-like nuclease domain can be modified or eliminated such that the Cas9-derived protein is unable to nick or cleave double stranded nucleic acid. In still other embodiments, all nuclease domains of the Cas9-derived protein can be modified or eliminated such that the Cas9-derived protein lacks all nuclease activity. Also, either the RuvC-like domain or the HNH-like domain can be inactivated independently of each other.

In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

### (III) Nucleic Acids Encoding RNA-Guided Endonucleases or Fusion Proteins

Another aspect of the present disclosure provides nucleic acids encoding any of the RNA-guided endonucleases or fusion proteins described above in sections (I) and (II), respectively. The nucleic acid can be RNA or DNA. In one embodiment, the nucleic acid encoding the RNA-guided endonuclease or fusion protein is mRNA. The mRNA can be 5′ capped and/or 3′ polyadenylated. In another embodiment, the nucleic acid encoding the RNA-guided endonuclease or fusion protein is DNA. The DNA can be present in a vector (see below).

The nucleic acid encoding the RNA-guided endonuclease or fusion protein can be codon optimized for efficient translation into protein in the cell, plant or animal of interest. For example, codons can be optimized for expression in humans, mice, rats, hamsters, cows, pigs, cats, dogs, fish, amphibians, plants, yeast, insects, and so forth. Programs for codon optimization are available as freeware. Commercial codon optimization programs are also available.

In some embodiments, DNA encoding the RNA-guided endonuclease or fusion protein can be operably linked to at least one promoter control sequence. In some iterations, the DNA coding sequence can be operably linked to a promoter control sequence for expression in the eukaryotic cell or animal of interest. The promoter control sequence can be constitutive, regulated, or tissue-specific. Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (EF1)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter. The promoter sequence can be wild type or it can be modified for more efficient or efficacious expression. In one exemplary embodiment, the encoding DNA can be operably linked to a CMV promoter for constitutive expression in mammalian cells.

In certain embodiments, the sequence encoding the RNA-guided endonuclease or fusion protein can be operably linked to a promoter sequence that is recognized by a phage RNA polymerase for in vitro mRNA synthesis. In such embodiments, the in vitro-transcribed RNA can be purified for use in the methods detailed below in sections (IV) and (V). For example, the promoter sequence can be a T7, T3, or SP6 promoter sequence or a variation of a T7, T3, or SP6 promoter sequence. In an exemplary embodiment, the DNA encoding the fusion protein is operably linked to a T7 promoter for in vitro mRNA synthesis using T7 RNA polymerase.

In alternate embodiments, the sequence encoding the RNA-guided endonuclease or fusion protein can be operably linked to a promoter sequence for in vivo expression of the RNA-guided endonuclease or fusion protein in bacterial or eukaryotic cells. In such embodiments, the expressed protein can be purified for use in the methods detailed below in sections (IV) and (V). Suitable bacterial promoters include, without limit, T7 promoters, lac operon promoters, trp promoters, variations thereof, and combinations thereof. An exemplary bacterial promoter is tac which is a hybrid of trp and lac promoters. Non-limiting examples of suitable eukaryotic promoters are listed above.

In additional aspects, the DNA encoding the RNA-guided endonuclease or fusion protein also can be linked to a polyadenylation signal (e.g., SV40 polyA signal, bovine growth hormone (BGH) polyA signal, etc.) and/or at least one transcriptional termination sequence. Additionally, the sequence encoding the RNA-guided endonuclease or fusion protein also can be linked to sequence encoding at least one nuclear localization signal, at least one cell-penetrating domain, and/or at least one marker domain, which are detailed above in section (I).

In various embodiments, the DNA encoding the RNA-guided endonuclease or fusion protein can be present in a vector. Suitable vectors include plasmid vectors, phagemids, cosmids, artificial/mini-chromosomes, transposons, and viral vectors (e.g., lentiviral vectors, adeno-associated viral vectors, etc.). In one embodiment, the DNA encoding the RNA-guided endonuclease or fusion protein is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel, et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In some embodiments, the expression vector comprising the sequence encoding the RNA-guided endonuclease or fusion protein can further comprise sequence encoding a gRNA. The sequence encoding the gRNA generally is operably linked to at least one transcriptional control sequence for expression of the gRNA in the cell or embryo of interest. For example, DNA encoding the gRNA can be operably linked to a promoter sequence that is recognized by RNA polymerase III (Pol III). Examples of suitable Pol III promoters include, but are not limited to, mammalian U6, U3, H1, and 7SL RNA promoters.

### (IV) Method for Modifying a Chromosomal Sequence Using an RNA-Guided Endonuclease

Another aspect of the present disclosure encompasses a method for modifying a chromosomal sequence in a eukaryotic cell or embryo. The method comprises introducing into a eukaryotic cell or embryo (i) at least one enzymatically inactivated RNA-guided endonuclease (or encoding nucleic acid) comprising at least one nuclear localization signal or nucleic acid encoding at least one RNA-guided endonuclease comprising at least one nuclear localization signal, (ii) at least one gRNA or DNA encoding at least one gRNA, and, optionally, (iii) at least one cytosine deaminase, (iv)uracil DNA glycosylase inhibitor (UGI). The method further comprises culturing the cell or embryo such that each gRNA directs an enzymatically inactivated RNA-guided endonuclease to a targeted site in the chromosomal sequence where the cytosine deaminase converts a cytosine to a uracil at the targeted site, and the uracil is repaired (i.e., converted) by a DNA repair process such that the chromosomal sequence is modified.

Accordingly, as discussed herein, the targeted chromosomal sequence can be modified or inactivated. For example, a single nucleotide change (SNP) can give rise to an altered protein product or introduce a “stop” codon into the reading frame of a coding sequence and thereby inactivate or “knock out” the sequence such that no protein product is made.

In other embodiments, the method can comprise introducing two (or more) RNA-guided endonucleases (or encoding nucleic acid) and two gRNAs (or encoding DNA) into a cell or embryo, wherein the RNA-guided endonucleases modify two cytosine bases. The cytosines can be within several base pairs, within tens of base pairs, or can be separated by many thousands of base pairs.

### (a) RNA-Guided Endonuclease

The method comprises introducing into a cell or embryo at least one RNA-guided endonuclease comprising at least one nuclear localization signal or nucleic acid encoding at least one RNA-guided endonuclease comprising at least one nuclear localization signal. Such RNA-guided endonucleases and nucleic acids encoding RNA-guided endonucleases are described above in sections (I) and (III), respectively. Such RNA-guided endonucleases may be preloaded with a gRNA.

In some embodiments, the RNA-guided endonuclease can be introduced into the cell or embryo as an isolated protein. In such embodiments, the RNA-guided endonuclease can further comprise at least one cell-penetrating domain, which facilitates cellular uptake of the protein. In other embodiments, the RNA-guided endonuclease can be introduced into the cell or embryo as an mRNA molecule. In still other embodiments, the RNA-guided endonuclease can be introduced into the cell or embryo as a DNA molecule. In general, DNA sequence encoding the fusion protein is operably linked to a promoter sequence that will function in the cell or embryo of interest. The DNA sequence can be linear, or the DNA sequence can be part of a vector. In still other embodiments, the fusion protein can be introduced into the cell or embryo as an RNA-protein complex comprising the fusion protein and the gRNA.

In alternate embodiments, DNA encoding the RNA-guided endonuclease can further comprise sequence encoding a gRNA. In general, each of the sequences encoding the RNA-guided endonuclease and the gRNA is operably linked to an appropriate promoter control sequence that allows expression of the RNA-guided endonuclease and the gRNA, respectively, in the cell or embryo. The DNA sequence encoding the RNA-guided endonuclease and the gRNA can further comprise additional expression control, regulatory, and/or processing sequence(s). The DNA sequence encoding the RNA-guided endonuclease and the gRNA can be linear or can be part of a vector

(b) Guide RNA (gRNA)

The method also comprises introducing into a cell or embryo at least one gRNA or DNA encoding at least one gRNA. A gRNA interacts with the enzymatically inactivated RNA-guided endonuclease to direct the endonuclease to a specific target site in the chromosomal sequence.

Each gRNA may comprise three regions: a first region that is complementary to the target site in the chromosomal sequence, a second internal region that forms a stem loop structure, and a third region that remains essentially single-stranded. The first region of each gRNA is different such that each gRNA guides a fusion protein to a specific target site. The second and third regions of each gRNA can be the same in all gRNAs.

The first region of the gRNA, termed the spacer, is complementary to sequence at the target site in the chromosomal sequence (i.e., protospacer sequence) such that the first region of the gRNA can base pair with the target site. In various embodiments, the first region of the gRNA can comprise from about 10 nucleotides to more than about 25 nucleotides. For example, the region of base pairing between the first region of the gRNA and the target site in the chromosomal sequence can be about 10, 11, 12, 13, 14, 15, 16, 17, 18, 19, 20, 22, 23, 24, 25, or more than 25 nucleotides in length. In an exemplary embodiment, the first region of the gRNA is about 19, 20, 21, 22 or 23 nucleotides in length.

The gRNA also comprises a second region that forms a secondary structure. In some embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The gRNA may also comprise a third region that remains essentially single-stranded. Thus, the third region has no complementarity to any chromosomal sequence in the cell of interest and has no complementarity to the rest of the gRNA. The length of the third region can vary. In general, the third region is more than about 4 nucleotides in length. For example, the length of the third region can range from about 5 to about 60 nucleotides in length.

The combined length of the second and third regions (also called the universal or scaffold region) of the gRNA can range from about 20 to about 120 nucleotides in length. In one aspect, the combined length of the second and third regions of the gRNA range from about 20 to about 100 nucleotides in length.

In some embodiments, the gRNA comprises a single molecule comprising all three regions, known as an sgRNA. In other embodiments, the gRNA can comprise two separate molecules. The first RNA molecule can comprise the first region of the gRNA and one half of the “stem” of the second region of the gRNA, known as the crRNA. The second RNA molecule can comprise the other half of the “stem” of the second region of the gRNA and the third region of the gRNA, known as the tracrRNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence to form a functional gRNA.

In some embodiments, the gRNA can be introduced into the cell or embryo as an RNA molecule. The RNA molecule can be transcribed in vitro. Alternatively, the RNA molecule can be chemically synthesized.

In other embodiments, the gRNA can be introduced into the cell or embryo as a DNA molecule. In such cases, the DNA encoding the gRNA can be operably linked to promoter control sequences for expression of the gRNA in the cell or embryo of interest. For example, the RNA coding sequence can be operably linked to a promoter sequence that is recognized by RNA polymerase III (Pol III). Examples of suitable Pol III promoters include, but are not limited to, mammalian U6 or H1 promoters. In exemplary embodiments, the RNA coding sequence is linked to a mouse or human U6 promoter. In other exemplary embodiments, the RNA coding sequence is linked to a mouse or human H1 promoter.

The DNA molecule encoding the gRNA can be linear or circular. In some embodiments, the DNA sequence encoding the gRNA can be part of a vector. Suitable vectors include plasmid vectors, phagemids, cosmids, artificial/mini-chromosomes, transposons, and viral vectors. In an exemplary embodiment, the DNA encoding the gRNA is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

In embodiments in which both the RNA-guided endonuclease and the gRNA are introduced into the cell as DNA molecules, each can be part of a separate molecule (e.g., one vector containing fusion protein coding sequence and a second vector containing gRNA coding sequence) or both can be part of the same molecule (e.g., one vector containing coding (and regulatory) sequence for both the fusion protein and the gRNA).

### (c) Target Site

An RNA-guided endonuclease in conjunction with a gRNA is directed to a target site in the chromosomal sequence, wherein the RNA-guided endonuclease (enzymatically inactivated) binds the chromosomal sequence. The target site has no sequence limitation except that the sequence is immediately adjacent to a consensus sequence. This consensus sequence is also known as a protospacer adjacent motif (PAM). Examples of PAM include, but are not limited to, NGG, NGGNG, and NNAGAAW (wherein N is defined as any nucleotide and W is defined as either A or T). As detailed above in section (IV)(b), the spacer region of the gRNA is complementary to the protospacer of the target sequence. Typically, the spacer region of the gRNA is about 19 to 21 nucleotides in length. Thus, in certain aspects, the sequence of the target site in the chromosomal sequence is 5′-N₁₉-2₁-NGG-3′.

The target site can be in the coding region of a gene, in an intron of a gene, in a control region of a gene, in a non-coding region between genes, etc. The gene can be a protein coding gene or an RNA coding gene. The gene can be any gene of interest.

### (d) Introducing Into the Cell or Embryo

The RNA-targeted endonuclease(s) (or encoding nucleic acid) and the gRNA(s) (or encoding DNA) can be introduced into a cell or embryo by a variety of means. In some embodiments, the cell or embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel, et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001). In other embodiments, the molecules are introduced into the cell or embryo by microinjection. Typically, the embryo is a fertilized one-cell stage embryo of the species of interest. For example, the molecules can be injected into the pronuclei of one cell embryos.

The RNA-targeted endonuclease(s) (or encoding nucleic acid), the gRNA(s) (or DNAs encoding the gRNA), and the optional uracil glycosylase inhibitor can be introduced into the cell or embryo simultaneously or sequentially. The ratio of the RNA-targeted endonuclease(s) (or encoding nucleic acid) to the gRNA(s) (or encoding DNA) generally will be about stoichiometric such that they can form an RNA-protein complex. In one embodiment, DNA encoding an RNA-targeted endonuclease and DNA encoding a gRNA are delivered together within the plasmid vector.

### (e) Culturing the Cell or Embryo

The method further comprises maintaining the cell or embryo under appropriate conditions such that the gRNA(s) directs the cytosine base editor fusion protein(s) to the targeted site(s) in the chromosomal sequence, and the cytosine deaminase introduces at least one cytosine-to-uracil conversion in the chromosomal sequence such that the chromosomal sequence is modified by a substitution of at least one nucleotide.

In embodiments in which a premature stop codon is introduced, the chromosomal sequence may be inactivated or “knocked out.” An inactivated protein-coding chromosomal sequence does not give rise to the protein coded by the wildtype chromosomal sequence.

In embodiments in which the chromosome sequence is modified without knocking out or inactivating the sequence, the protein encoded may be either 1) modified resulting in a protein with lesser or greater function or 2) in the case of correcting natural mutations, brought back to or approximate wildtype sequence and/or function. In general, the cell is maintained under conditions appropriate for cell growth and/or maintenance. Suitable cell culture conditions are well known in the art and are described, for example, in Santiago, et al., (2008) PNAS 105:5809-5814; Moehle, et al., (2007) PNAS 104:3055-3060; Urnov, et al., (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow for editing to take place. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal derived from the embryo. Such an animal would comprise the modified chromosomal sequence in every cell of the body.

### (f) Cell and Embryo Types

A variety of eukaryotic cells and embryos are suitable for use in the method. For example, the cell can be a human cell, a non-human mammalian cell, a non-mammalian vertebrate cell, an invertebrate cell, an insect cell, a plant cell, a yeast cell, a single cell eukaryotic organism, or a prokaryote. In general, the embryo is a non-human mammalian embryo. In specific embodiments, the embryos can be a one cell non-human mammalian embryo. Exemplary mammalian embryos, including one cell embryos, include without limit mouse, rat, hamster, rodent, rabbit, feline, canine, ovine, porcine, bovine, equine, and nonhuman primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary embodiments, the cell is a mammalian cell.

Non-limiting examples of suitable mammalian cells include Chinese hamster ovary (CHO) cells, baby hamster kidney (BHK) cells; mouse myeloma NSO cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T½ cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells. An extensive list of mammalian cell lines may be found in the American Type Culture Collection catalog (ATCC, Manassas, Va.).

### (V) Method for Using a Fusion Protein to Modify a Chromosomal Sequence or Regulate Expression of a Chromosomal Sequence

Another aspect of the present disclosure encompasses a method for modifying a chromosomal sequence or regulating expression of a chromosomal sequence in a cell or embryo. The method comprises introducing into the cell or embryo (a) at least one cytosine base editor fusion protein or nucleic acid encoding at least one fusion protein, wherein the fusion protein comprises a CRISPR/Cas-like protein or a fragment thereof and an effector domain or tethered protein encoding a cytosine deaminase, and (b) at least one gRNA or DNA encoding the gRNA, wherein the gRNA guides the CRISPR/Cas-like protein of the fusion protein to a targeted site in the chromosomal sequence and the cytosine deaminase of the fusion protein modifies the chromosomal sequence or regulates expression of the chromosomal sequence by modification of a regulatory sequence associated with the chromosomal sequence.

Fusion proteins comprising a CRISPR/Cas-like protein or a fragment thereof and an effector domain are detailed above in section (II). In general, the fusion proteins disclosed herein further may comprise at least one nuclear localization signal. Nucleic acids encoding fusion proteins are described above in section (III). In some embodiments, the fusion protein can be introduced into the cell or embryo as an isolated protein (which can further comprise a cell-penetrating domain). Furthermore, the isolated fusion protein can be part of a protein-RNA complex comprising the gRNA. In other embodiments, the fusion protein can be introduced into the cell or embryo as a RNA molecule (which can be capped and/or polyadenylated). In still other embodiments, the fusion protein can be introduced into the cell or embryo as a DNA molecule. For example, the fusion protein and the gRNA can be introduced into the cell or embryo as discrete DNA molecules or as part of the same DNA molecule. Such DNA molecules can be plasmid vectors.

### (VI) Genetically Modified Cells and Animals

The present disclosure encompasses genetically modified cells, non-human embryos, and non-human animals comprising at least one chromosomal sequence that has been modified using an enzymatically inactivated RNA-guided endonuclease-mediated or fusion protein-mediated process, for example, using the methods described herein. The disclosure provides cells comprising at least one DNA or RNA molecule encoding an RNA-guided endonuclease or fusion protein targeted to a chromosomal sequence of interest or a fusion protein, at least one gRNA, and optionally one or more free standing or fused uracil glycosylase inhibitors (UGI). The disclosure also provides non-human embryos comprising at least one DNA or RNA molecule encoding an enzymatically inactivated RNA-guided endonuclease or fusion protein targeted to a chromosomal sequence of interest, at least one gRNA, and optionally one or more UGIs.

The present disclosure provides genetically modified non-human animals, non-human embryos, or animal cells comprising at least one modified chromosomal sequence. The modified chromosomal sequence may be modified such that it is (1) inactivated or (2) has an altered expression or produces an altered protein product. The chromosomal sequence is modified with an RNA guided endonuclease-mediated or fusion protein-mediated process, using the methods described herein.

As discussed, one aspect of the present disclosure provides a genetically modified animal in which at least one chromosomal sequence has been modified. In one embodiment, the genetically modified animal comprises at least one inactivated chromosomal sequence. The modified chromosomal sequence may be inactivated such that the sequence is not transcribed and/or a functional protein product is not produced. Thus, a genetically modified animal comprising an inactivated chromosomal sequence may be termed a “knock out.” As a consequence of the mutation, the targeted chromosomal sequence is inactivated and a functional protein is not produced. The inactivated chromosomal sequence comprises no exogenously introduced sequence. Also included herein are genetically modified animals in which two, three, four, five, six, seven, eight, nine, or ten or more chromosomal sequences are inactivated.

In another embodiment, the modified chromosomal sequence can be altered such that it codes for a variant protein product. For example, a genetically modified animal comprising a modified chromosomal sequence can comprise a targeted point mutation(s) or other modification such that an altered protein product is produced. In one embodiment, the chromosomal sequence can be modified such that at least one nucleotide is changed and the expressed protein comprises one changed amino acid residue (missense mutation). In another embodiment, the chromosomal sequence can be modified to comprise more than one missense mutation such that more than one amino acid is changed. The altered or variant protein can have altered properties or activities compared to the wildtype protein (or as compared to an unmodified protein in the case of correcting a natural genetic mutation), such as altered substrate specificity, altered enzyme activity, altered kinetic rates, etc.

In another embodiment, the genetically modified animal can comprise at least one genetic change where a non-expressed gene may be activated, which is termed a “knock in.” The chromosomally modified sequence can, for example, encode a protein resembling an orthologous protein, an endogenous protein, or combinations of both.

In yet another embodiment, the genetically modified animal can comprise at least one modified chromosomal sequence encoding a protein such that the expression pattern of the protein is altered. For example, regulatory regions controlling the expression of the protein, such as a promoter or a transcription factor binding site, can be altered such that the protein is over-produced, or the tissue-specific or temporal expression of the protein is altered, or a combination thereof. Alternatively, the expression pattern of the protein can be altered in combination with using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence. The genetically modified animal comprising the lox-flanked chromosomal sequence can then be crossed with another genetically modified animal expressing Cre recombinase. Progeny animals comprising the lox-flanked chromosomal sequence and the Cre recombinase are then produced, and the lox-flanked chromosomal sequence is recombined, leading to deletion or inversion of the chromosomal sequence encoding the protein. Expression of Cre recombinase can be temporally and conditionally regulated to effect temporally and conditionally regulated recombination of the chromosomal sequence.

In any of these embodiments, the genetically modified animal disclosed herein can be heterozygous for the modified chromosomal sequence. Alternatively, the genetically modified animal can be homozygous for the modified chromosomal sequence.

The genetically modified animals disclosed herein can be crossbred to create animals comprising more than one modified chromosomal sequence or to create animals that are homozygous for one or more modified chromosomal sequences. For example, two animals comprising the same modified chromosomal sequence can be crossbred to create an animal homozygous for the modified chromosomal sequence. Alternatively, animals with different modified chromosomal sequences can be crossbred to create an animal comprising both modified chromosomal sequences.

In other embodiments, an animal comprising a modified chromosomal sequence can be crossbred to combine the modified chromosomal sequence with other genetic backgrounds. By way of non-limiting example, other genetic backgrounds may include wild-type genetic backgrounds, genetic backgrounds with deletion mutations, genetic backgrounds with a targeted integration, and genetic backgrounds with non-targeted integrations.

The term “animal,” as used herein, refers to a human or non-human animal. The animal may be an embryo, a juvenile, or an adult. Suitable animals include vertebrates such as mammals, birds, reptiles, amphibians, shellfish, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In one embodiment, the animal is not a genetically modified mouse. In each of the foregoing iterations of suitable animals for the invention, the animal does not include exogenously introduced, randomly integrated transposon sequences.

A further aspect of the present disclosure provides genetically modified cells or cell lines comprising at least one modified chromosomal sequence. The genetically modified cell or cell line can be derived from any of the genetically modified animals disclosed herein. Alternatively, the chromosomal sequence can be modified in a cell as described herein above (in the paragraphs describing chromosomal sequence modifications in animals) using the methods descried herein. The disclosure also encompasses a lysate of said cells or cell lines.

In preferred embodiments, the cells are eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells can be primary cells. The cells may be of a variety of cell types, e.g., fibroblast, myoblast, T or B cell, macrophage, epithelial cell, and so forth.

The cells can be eukaryotic cells or prokaryotic cells. For example, cells from bacteria, protists, plants, animals, fungi are contemplated. Animals may include, but are not limited to, insects and mammals.

When mammalian cell lines are used, the cell line can be any established cell line or primary cell type, or one that is not yet described. The cell line can be adherent or non-adherent, or the cell line can be grown under conditions that encourage adherent, non-adherent or organotypic growth using standard techniques known to individuals skilled in the art. Non-limiting examples of suitable mammalian cells and cell lines are provided herein in section (IV)(g). In still other embodiments, the cell can be a stem cell. Non-limiting examples of suitable stem cells are provided in section (IV)(g).

The present disclosure also provides a genetically modified non-human embryo comprising at least one modified chromosomal sequence. The chromosomal sequence can be modified in an embryo as described herein above (in the paragraphs describing chromosomal sequence modifications in animals) using the methods descried herein. In one embodiment, the embryo is a non-human fertilized one-cell stage embryo of the animal species of interest. Exemplary mammalian embryos, including one cell embryos, include without limit, mouse, rat, hamster, rodent, rabbit, feline, canine, ovine, porcine, bovine, equine, and primate embryos.

Further, non-mammalian cells and cell lines may be used including, but not limited to, plant, insect and prokaryote cells and cell lines, as are known to one of skill in the art. Such cells may be newly developed for a specific purpose or may be readily available such as from ATCC (Bethesda, MD) and other sources known to one of skill in the art. In short, the compositions and methods of the present invention should be useful for base editing in any cell having nucleic acids and Cas-based repair pathways including viruses after infection into a host cell.

### (VII) Kits

Still another aspect of the present disclosure provides kits for carrying out the methods described above.

The kits provided herein generally include instructions for carrying out the processes detailed above. Instructions included in the kits may be affixed to packaging material, may be included as a package insert or as a downloadable file. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

As various changes could be made in the above-described processes and kits without departing from the scope of the invention, it is intended that all matter contained in the above description and in the examples given below, shall be interpreted as illustrative and not in a limiting sense.

### EXEMPLIFICATION

### Example 1

CBE with an N-Terminal SSB Fusion Yields a Narrower Editing Window than a CBE with No SSB Domain

Cytosine base editor (CBE) proteins were constructed by fusing human APOBEC3A or the C-terminal domain of human APOBEC3B to the amino-terminus of an SpCas9 nickase protein and two uracil glycosylase inhibitor (UGI) domains to the C-terminus (SEQ ID NO: 4 and 5). The novel CBE proteins were constructed by fusing a single-stranded DNA binding protein (SSB) domain from T4 or T7 bacteriophage to the amino termini of the CBEs described above (SEQ ID NO: 6-9). All proteins were expressed and purified from E. coli BL21AI by autoinduction and nickel column chromatography and were stored at −80° C. before use in a buffer containing 10% glycerol, 300 mM KCl, 20 mM HEPES (pH 7.5), and 1 mM DTT. Synthetic single guide RNAs (sgRNAs) targeting four human genomic sites were purchased from MilliporeSigma. Spacer sequences of the sgRNAs are given in Table 1. Each experimental condition was tested in two technical replicates.

TABLE 1
sgRNA Spacer Sequences and PCR Primer Sequences

| | Sequence (5′ - 3′) | SEQ ID NO. |
|---|---|---|
| EMX1-15 spacer | GCTCCCATCACATCAACCGG | 12 |
| HBB03 spacer | CTTGCCCCACAGGGCAGTAA | 13 |
| HEKSite2 spacer | GAACACAAAGCATAGACTGC | 14 |
| RNF2 spacer | GTCATCTTAGTCATTACCTG | 15 |
| EMX1-15 PCR forward | TCGTCGGCAGCGTCAGATGTGTATAAGAGACAGNNNNNNCCTGAGTTTCTCATCTGTGCCC | 16 |
| EMX1-15 PCR reverse | GTCTCGTGGGCTCGGAGATGTGTATAAGAGACAGNNNNNNTGACTCCAGGCCTCCCCAAA | 17 |
| HBB03 PCR forward | TCGTCGGCAGCGTCAGATGTGTATAAGAGACAGNNNNNNGCCAATCTACTCCCAGGAGC | 18 |
| HBB03 PCR reverse | GTCTCGTGGGCTCGGAGATGTGTATAAGAGACAGNNNNNNAGGCAGAGAGAGTCAGTGCCTA | 19 |
| HEKSite2 PCR forward | TCGTCGGCAGCGTCAGATGTGTATAAGAGACAGNNNNNNTGAACTTCCCAAGTGAGAAGCCAG | 20 |
| HEKSite2 PCR reverse | GTCTCGTGGGCTCGGAGATGTGTATAAGAGACAGNNNNNNTGGCAGGACGTCTGCCCAAT | 21 |
| RNF2 PCR forward | TCGTCGGCAGCGTCAGATGTGTATAAGAGACAGNNNNNNTGTTAGCCAACATACAGAAGTCAG | 22 |
| | GAATGC | |
| RNF2 PCR reverse | GTCTCGTGGGCTCGGAGATGTGTATAAGAGACAGNNNNNNTTCAGACCATAGCACTTCCCTTCC | 23 |

Ribonucleoprotein (RNP) complexes were prepared by incubating for 15 min at room temperature: 15 μg of CBE protein and 200 pmols sgRNA in buffer (20 mM HEPES, 100 mM KCl, 0.5 mM DTT, 0.1 mM EDTA, pH 7.5) for a final volume of 10 μL. The molar ratio of CBE to sgRNA was approximately 1:3. RNPs were kept on ice until transfection. HEK293 cells were obtained from ATCC and grown at 37° C. and 5% CO₂ in DMEM supplemented with 10% FBS, 2 mM L-glutamine, 1 mM sodium pyruvate, and 0.1 mM non-essential amino acids. Cells were seeded at 1.67×10⁴ cells/cm² of tissue culture surface area two days before transfection. At the time of transfection, cells were trypsinized to obtain a single-cell suspension, washed twice with Hank's Balanced Salt Solution and resuspended in Nucleofector Solution V (Lonza, Basel, CH) at 2.5×10⁵ cells per 100 μL. Nucleofection was performed by mixing 100 μL of prepared cell suspension with 10 μL of complexed CBE RNP by pipetting up and down six times before transferring to a cuvette for electroporation using program Q-001 on a Nucleofector 2b machine. Nucleofected cells were immediately transferred to 6-well plates containing 2 mL pre-warmed media per well and grown for 3 days before harvest.

Genomic DNA was harvested by trypsinizing transfected cells and resuspending in 75 μL QuickExtract reagent (Lucigen). Suspensions were incubated at 60° C. for 15m and 95° C. for 15m. Genomic regions targeted by the CBE were amplified by PCR using JumpStart Taq ReadyMix (MilliporeSigma, Burlington, MA) and the following cycling conditions: 94° C./2m; 25 cycles of 94° C./30s, 62° C./30s, 72° C./45s; 72° C./5m. Primers are listed in Table 1. PCR products underwent a second round of amplification using Illumina index primers and JumpStart Taq ReadyMix and the following conditions: 95° C./3m; 9 cycles of 95° C./30s, 55° C./30s, 72° C./30s; 72° C./5m. Indexed PCR products were purified by Select-α-Size DNA Clean & Concentrator MagBeads (Zymo, Irvine, CA), using 1.2× beads by volume, quantified by PicoGreen (ThermoFisher, Waltham, MA), and pooled according to DNA content. Pools were diluted to 4 nM. Sequencing was performed on an Illumina MiSeq instrument using a 300-cycle kit to obtain single-end reads. FASTQ files for each sample were analyzed using a custom analysis script.

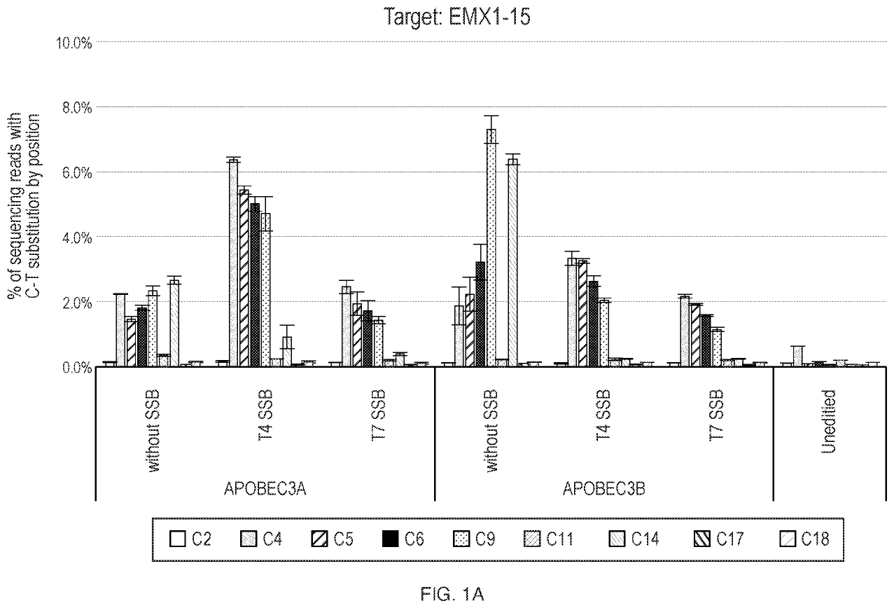

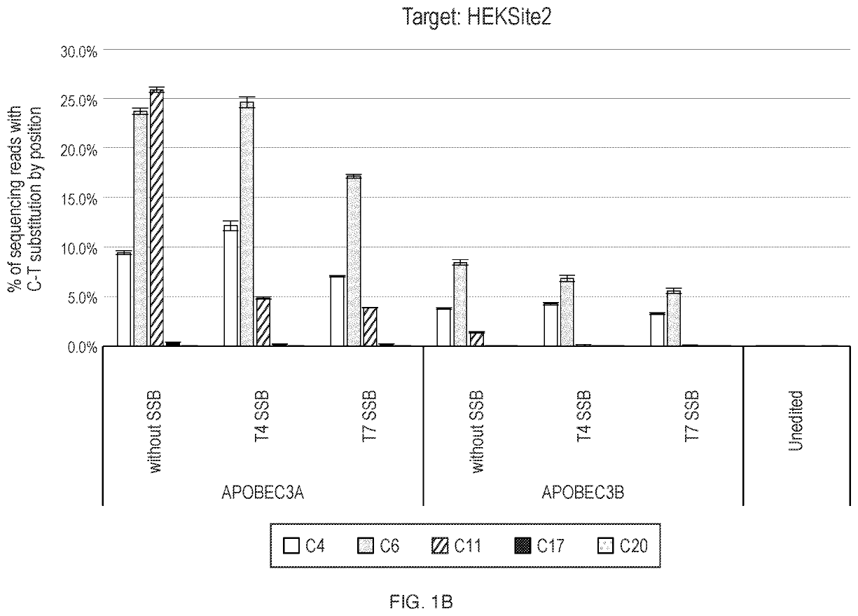

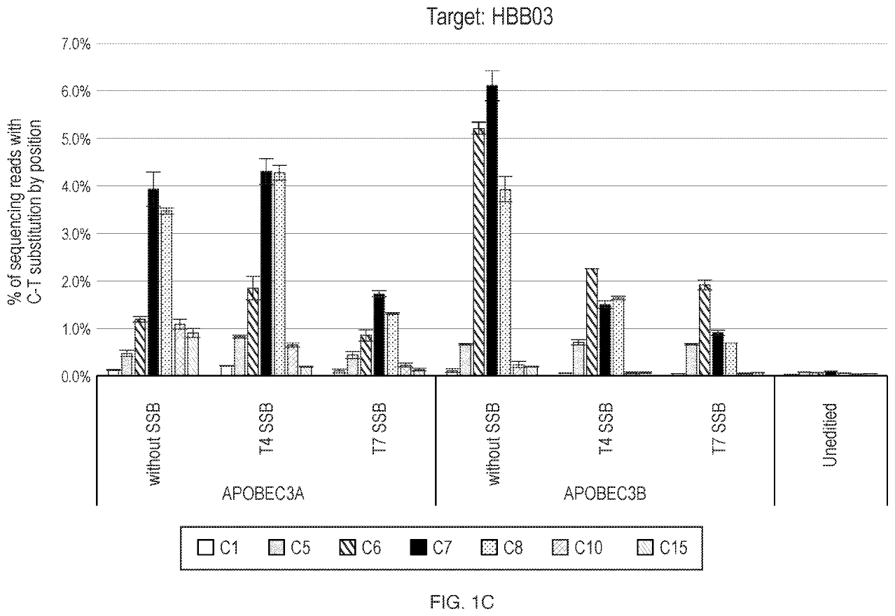

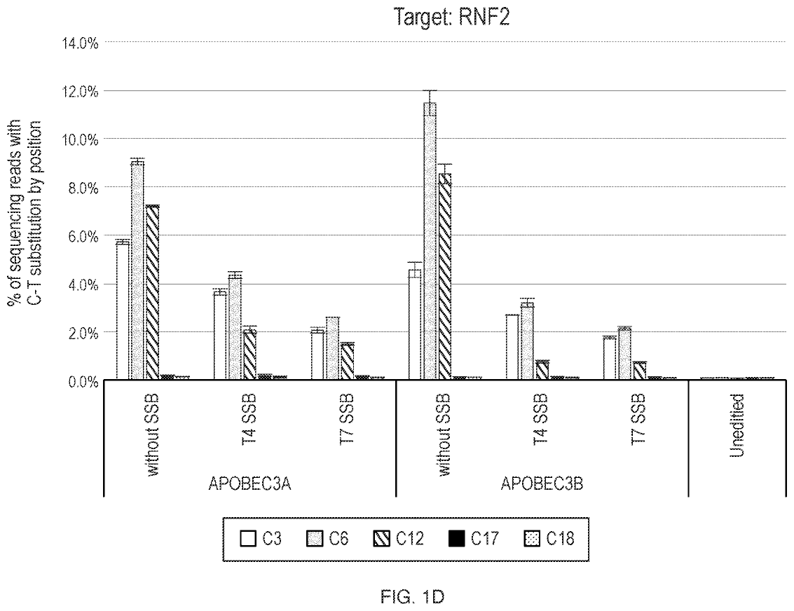

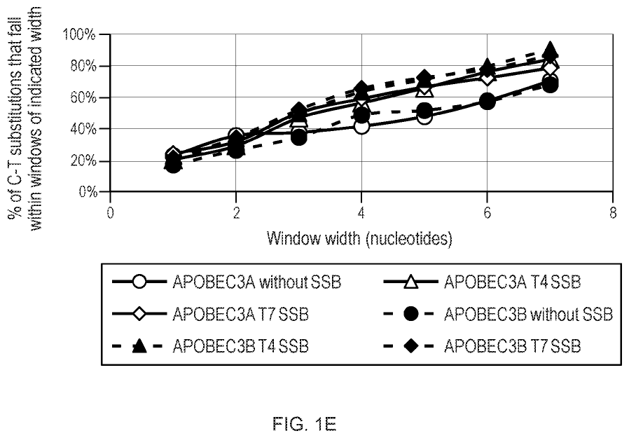

Results are presented in FIG. 1. In FIGS. 1A-1D, the percentage of sequencing reads containing C-to-T substitutions at each cytosine residue in individual target sites is plotted. Positions are indicated by their distance from the 5′ end of the target sequence. Values are average±standard deviation for two replicates. All transfections were performed with the same cells, on the same day. The results show that absolute and relative editing efficiency is target-specific. Editing profiles demonstrate a shift in editing window for CBE variants with N-terminal SSB fusion away from the PAM relative to the editing window for CBE lacking the SSB fusion.

In FIG. 1E, the percentage of C-to-T substitutions that fall within windows of given widths is plotted against the width of that window for each variant tested. The results show that CBE variants with N-terminal SSB fusions (solid and open diamonds and solid and open triangles) have narrower editing windows than CBE variants without the SSB fusions (solid and open circles), regardless of whether the deaminase used was APOBEC3A (open symbols, solid lines) or APOBEC3B (filled symbols, dashed lines). Variants with N-terminal SSB fusion generate 50% of C-to-T substitutions within a 4 nt window, whereas variants without SSB require at least 5 nt to contain the same fraction of edits. Further, CBE variants with N-terminal SSB fusion generate 67% of C-to-T substitutions in a window of approximately 5 nt, whereas CBE variants lacking SSB require a 7 nt window to contain the same fraction of edits.

### Example 2

CBE RNPs without 2×UGI have Higher Editing Activity than CBE RNPs with C-Terminal 2×UGI Fusion

A minimal cytosine base editor (CBE) protein without UGI was constructed by fusing the C-terminal domain of human APOBEC3B to the amino terminus of an SpCas9 nickase protein (SEQ ID NO: 10). The minimal CBE was further modified to contain a T4 phage SSB at the amino terminus of the CBE (SEQ ID NO: 11). CBE proteins with 2×UGI used in this example are as described in Example 1. Proteins were expressed and purified from E. coli as in Example 1. Synthetic single guide RNAs (sgRNAs) targeting four human genomic sites were purchased from MilliporeSigma. Spacer sequences of the sgRNAs are given in Table 1. Each experimental condition was tested in two technical replicates. Ribonucleoprotein (RNP) complexes were prepared by incubating for 15 min at room temperature: 40 pmol of CBE protein and 120 pmol sgRNA in buffer (20 mM HEPES, 100 mM KCl, 0.5 mM DTT, 0.1 mM EDTA, pH 7.5) for a final volume of 10 μL. RNPs were kept on ice until transfection. Transfections were performed in HEK293 cells as described in Example 1. Genomic DNA was purified, genomic target sites were amplified, and libraries were prepared, sequenced, and analyzed as described in Example 1.

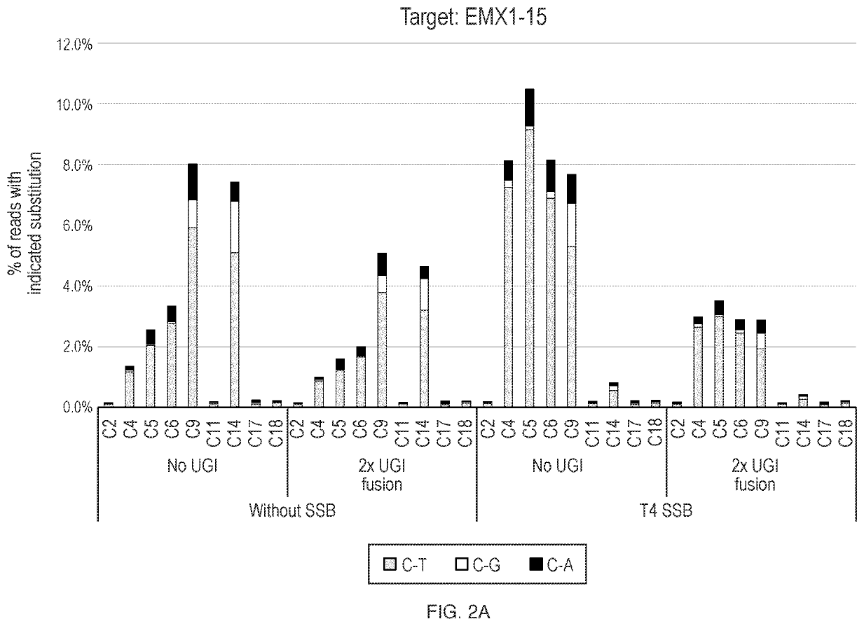

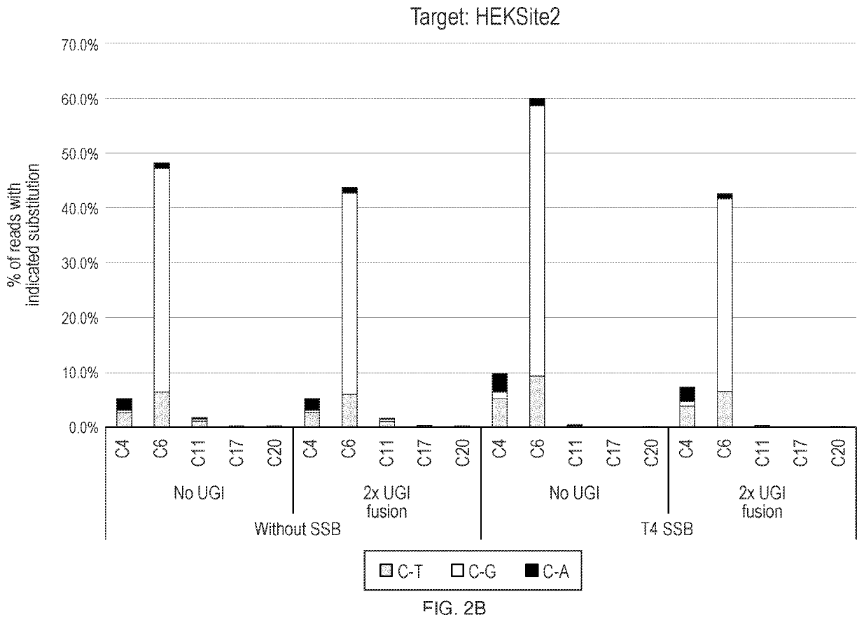

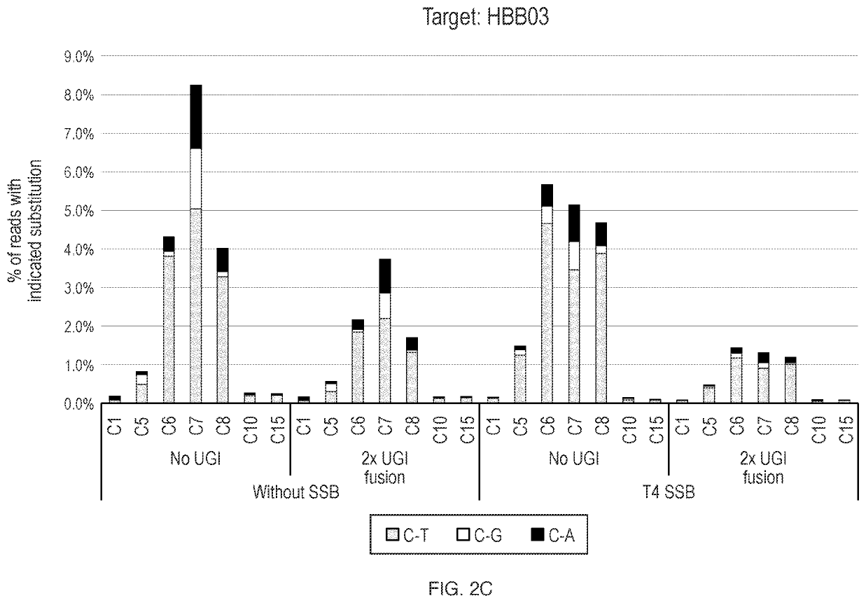

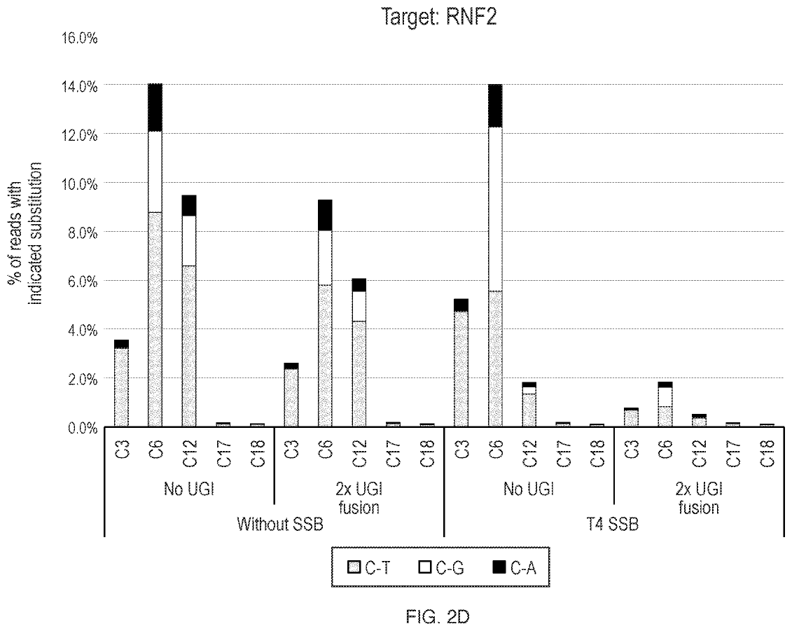

Results are presented in FIG. 2A-2D. The percentage of sequencing reads containing substitutions at each cytosine residue of the protospacer is plotted for CBE variants with and without the 2×UGI fusion. The results show that CBE variants lacking UGI as part of the fusion protein have higher substitution rates than CBE variants containing 2×UGI for all targets tested. Editing rates for the CBE variants without UGI were up to 3.7-fold higher than rates for the 2×UGI variants. The proportion of substitutions that are C-to-T are similar for CBE variants with and without the 2×UGI fusion, suggesting that the fused UGI domains do not function to limit C-to-A and C-to-G substitutions as seen in the plasmid delivery format.

### Example 3

Dose-Dependent Reduction in Indel Formation and C-A and C-G Substitution with the Co-Transfection of Uracil Glycosylase Inhibitor Protein and CBE RNP

Cytosine base editor (CBE) proteins were constructed by fusion of the C-terminal domain of human APOBEC3B to the amino-terminus of an SpCas9 nickase protein and two uracil glycosylase inhibitor (UGI) domains to the C-terminus. This CBE further contains a single-stranded DNA binding protein (SSB) domain from T4 bacteriophage fused to the N-terminus for a final structure of N-SSB-deaminase-nCas9-C, where N and C denote the amino- and carboxyl-terminus of the protein as described in Example 1. A recombinant UGI containing a Bacillus phage UGI, a c-MYC nuclear localization signal (NLS), and a SV40 Large T antigen NLS (SEQ ID NO: 3) was purified from E. coli. CBE proteins were purified from E. coli as in Example 1. Synthetic single guide RNAs (sgRNAs) targeting four human genomic sites were purchased from MilliporeSigma. Spacer sequences of the sgRNAs are given in Table 1. Each experimental condition was tested in two technical replicates.

Ribonucleoprotein (RNP) complexes were prepared by incubating for 15 min at room temperature: 15 μg of CBE protein, 200 pmol of sgRNA, and 0-15 μg (0-1185 μmol) UGI in buffer (20 mM HEPES, 100 mM KCl, 0.5 mM DTT, 0.1 mM EDTA, pH 7.5) for a final volume of 10 μL. The molar ratio of CBE to sgRNA was approximately 1:3. RNPs were kept on ice until transfection. Transfections were performed in HEK293 cells as described in Example 1. Genomic DNA was purified, genomic target sites were amplified, and libraries were prepared and sequenced as described in Example 1. FASTQ files for each sample were analyzed using a custom analysis script and the CRIS.py software package (Connelly & Pruett-Miller, Sci. Rep., 2019).

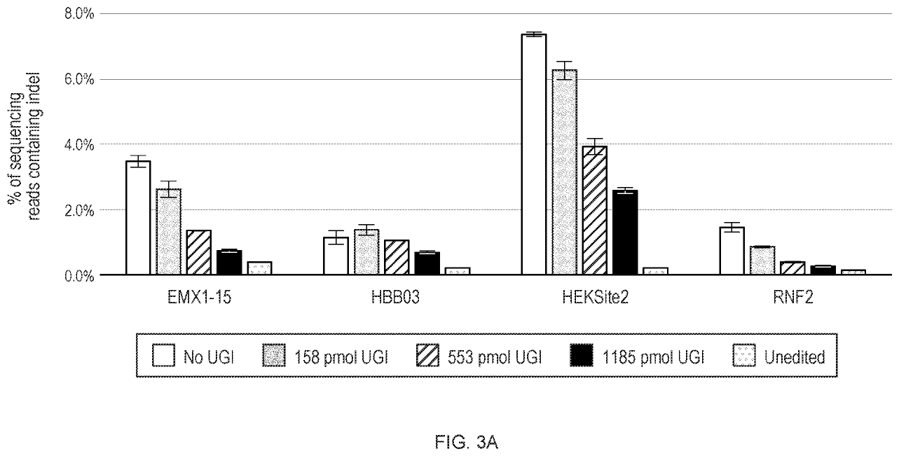

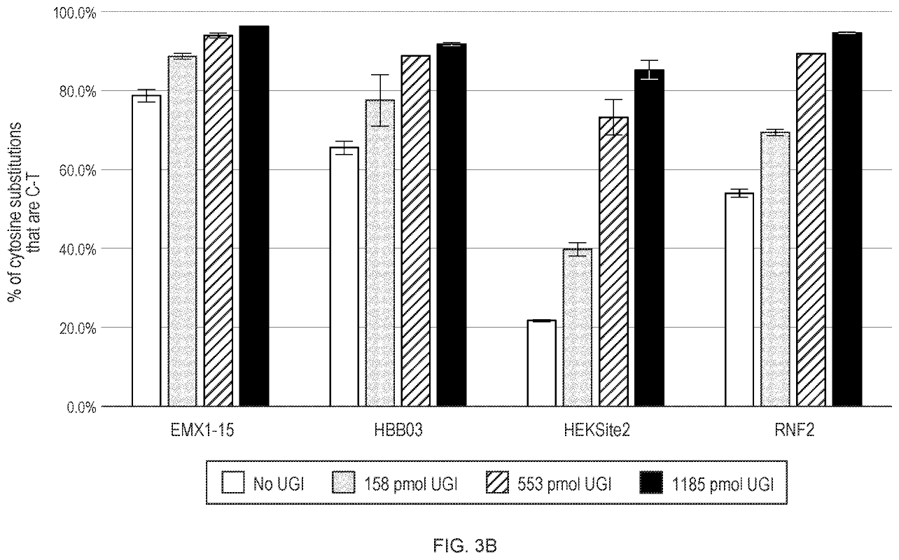

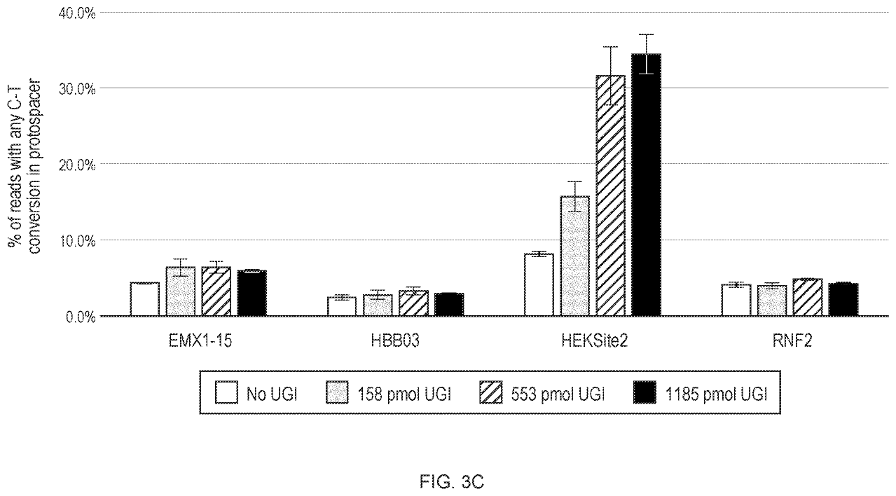

Results are presented in FIG. 3. In FIG. 3A, the percentage of sequencing reads containing insertions or deletions is plotted for four targets edited with CBE RNPs. Values are average±standard deviation for two replicates. Co-transfection of UGI protein alongside the RNP complexes results in a dose dependent decrease in the indel rate. At the highest dose of UGI, indel rates are at least 40% lower (target HBB03) than control transfections without UGI, and up to 80% lower (targets EMX1-15 and RNF2) than the control.

In FIG. 3B, the percentage of cytosine substitutions that are C-to-T is plotted. Values are average±standard deviation for two replicates. Co-transfection of UGI protein alongside the RNP complexes results in a dose-dependent increase in the proportion of cytosine substitutions that are C-to-T, and therefore a decrease in the proportion of undesired C-to-A and C-to-G substitutions. With the highest dose of UGI, the C-to-T proportion was at least 20% higher (EMX1-15) than control transfections without UGI, and up to 4-fold higher than the control (HEKSite2).

In FIG. 3C, the percentage of reads containing at least one C-to-T substitution within the protospacer is plotted. Values are average±standard deviation for two replicates. Co-transfection of UGI protein alongside the RNP complexes, minimally, does not decrease the rate of C-to-T editing. For target HEKSite2, co-transfection of UGI protein results in a dose-dependent increase in the rate of C-to-T editing concomitant with the increased proportion of substitutions that are C-to-T. This demonstrates that the reduction in C-to-A and C-to-G substitutions, as well as the reduction in indels, is not due to reduced editing activity.

### Example 4

Addition of Dextran Sulfate to the Co-Transfection of Uracil Glycosylase Inhibitor Protein with CBE RNP Enhances the Rate of C-T Substitution

Cytosine base editor (CBE) proteins were constructed, minimally, by fusing the C-terminal domain of human APOBEC3B to the amino-terminus of an SpCas9 nickase protein. One CBE variant used in this example was constructed by further fusing the single-stranded DNA binding protein (SSB) domain from T4 bacteriophage to the N-terminus of the CBE described above. CBE and Bacillus phage uracil glycosylase inhibitor (UGI) proteins were expressed and purified from E. coli as in Examples 1 and 3, respectively. Synthetic single guide RNAs (sgRNAs) targeting four human genomic sites were purchased from MilliporeSigma. Spacer sequences of the sgRNAs are given in Table 1. Each experimental condition was tested in two technical replicates.

Ribonucleoprotein (RNP) complexes were prepared by incubating for 15 min at room temperature: CBE protein, sgRNA, and 0 or 7 μg UGI in buffer (20 mM HEPES, 100 mM KCl, 0.5 mM DTT, 0.1 mM EDTA, pH 7.5) for a final volume of 10 μL. For the CBE variant without SSB, 80 pmol protein and 200 pmol sgRNA were used; for the CBE variant containing SSB, 40 pmol protein and 120 pmol sgRNA were used. RNPs were kept on ice until transfection.

Dextran sulfate sodium salt with average molecular weight greater than 500 kDa (Product number: D8906) was purchased from MilliporeSigma. A dextran sulfate solution was prepared by dissolving the chemical in water at 50 μg/μL and sterilized by filtration through a 0.22 um filter. The stock solution was diluted with water to prepare working solutions of 1 μg/μL.

HEK293 cells were obtained from ATCC and grown at 37° C. and 5% CO₂ in DMEM supplemented with 10% FBS, 2 mM L-glutamine, 1 mM sodium pyruvate, and 0.1 mM non-essential amino acids. Cells were seeded at 1.67×10⁴ cells/cm² of tissue culture surface area two days before transfection. Cells were trypsinized to obtain a single-cell suspension, washed twice with Hank's Balanced Salt Solution and resuspended in Nucleofector Solution V (Lonza) at approximately 2.5×10⁵ cells per 100 μL. Dextran sulfate solution was added to the cell suspension to a final concentration of 0.5 μg per 100 μL and mixed well by swirling. Nucleofection was performed by mixing 100 μL of prepared cell suspension with 10 μL of complexed CBE RNP by pipetting up and down six times before transferring to a cuvette for electroporation using program Q-001 on the Nucleofector 2b machine. Nucleofected cells were immediately transferred to 6-well plates containing 2 mL pre-warmed media per well and grown for 3 days before harvest.

Genomic DNA was purified, genomic target sites were amplified, and libraries were prepared and sequenced as described in Example 1. FASTQ files for each sample were analyzed using a custom analysis script and the BE-Analyzer web-based analysis tool (Hwang et al., BMC Bioinformatics, 2018).

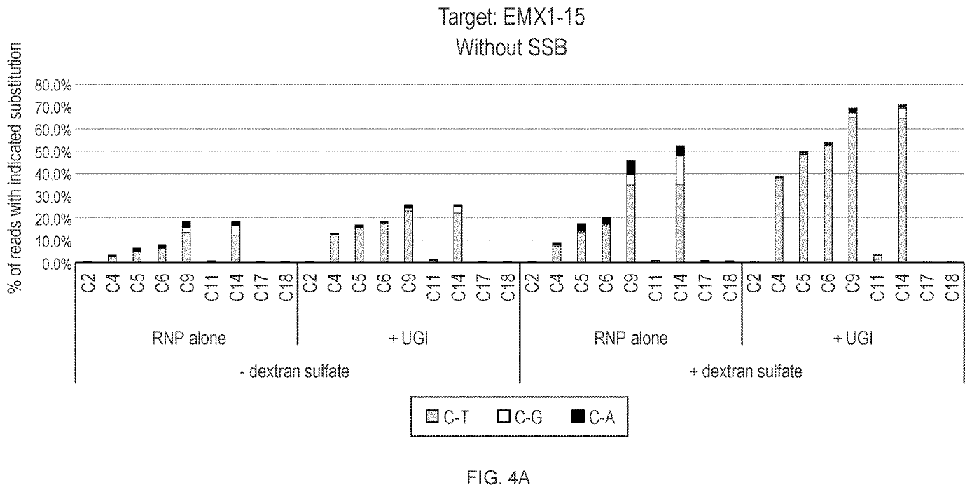

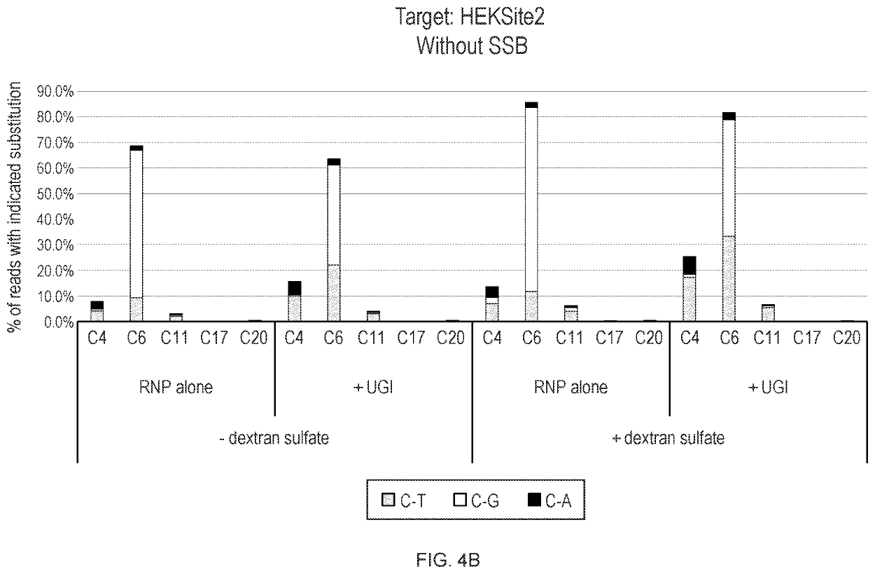

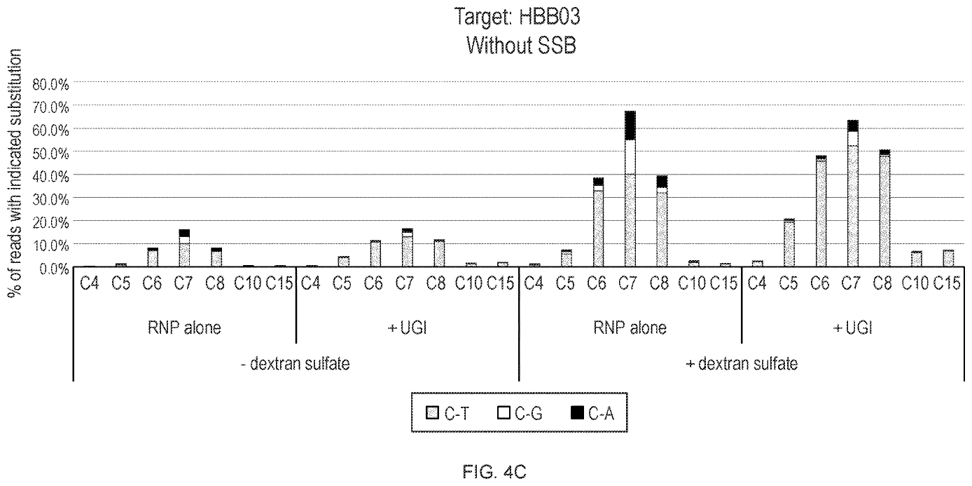

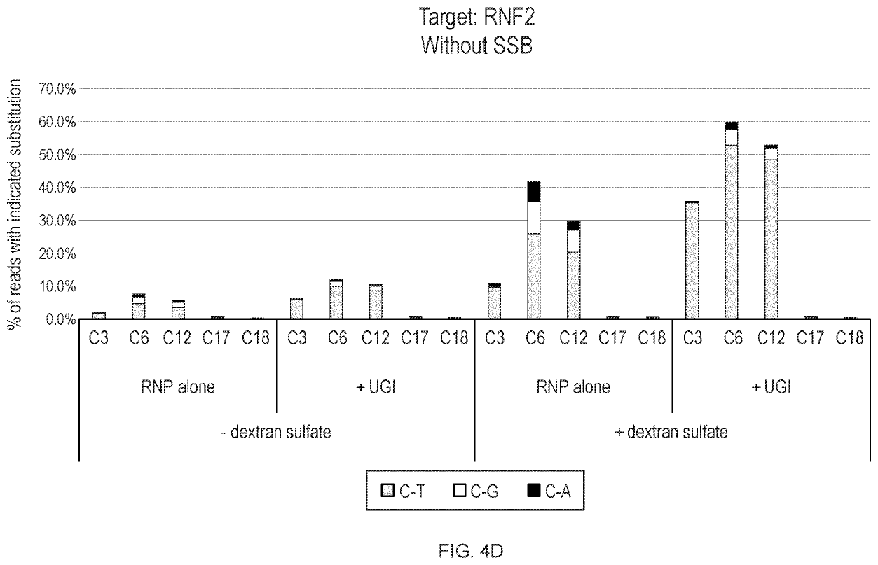

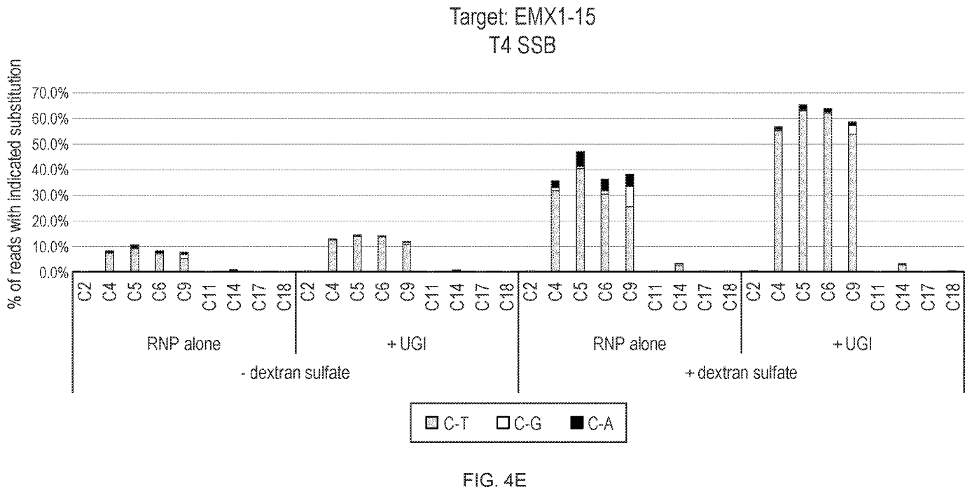

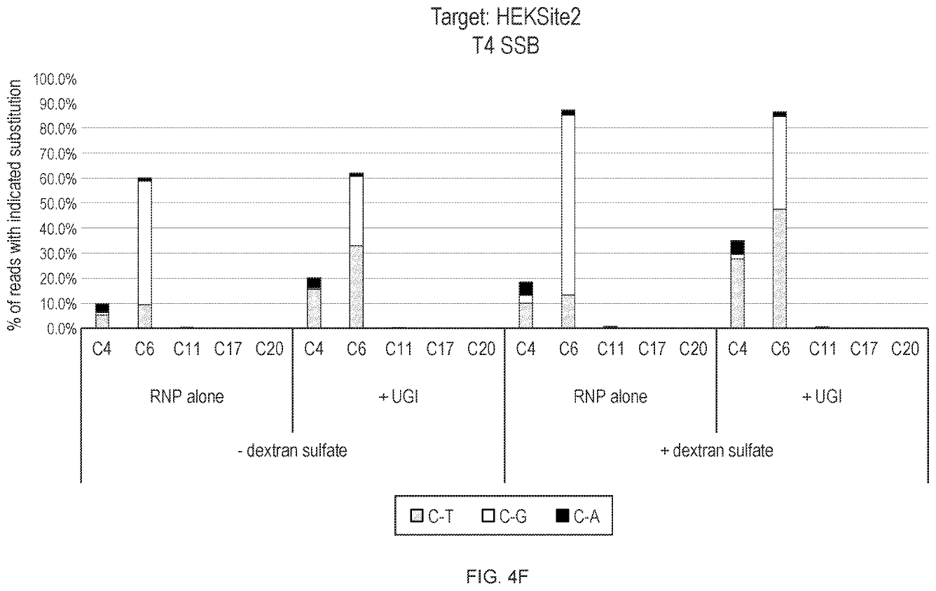

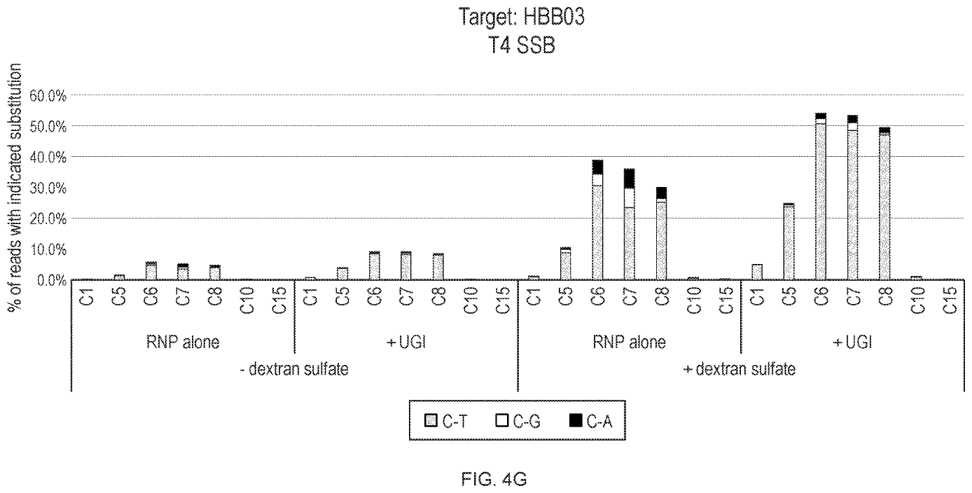

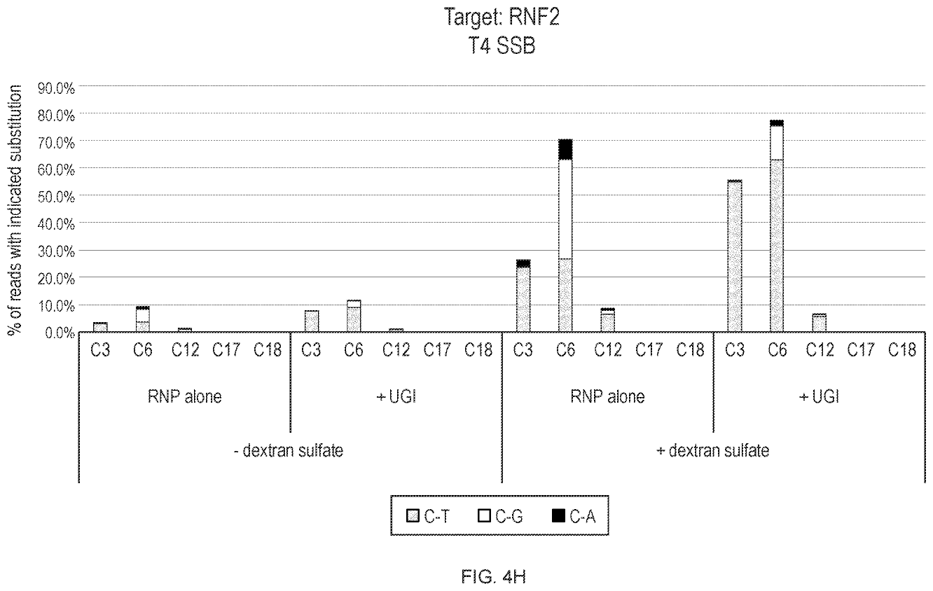

Results are presented in Table 2 and FIG. 4. Table 2 provides the percentage of reads with any C-to-T substitution for each CBE variant, each target and each transfection condition, as determined using BE-Analyzer. Values are average±standard deviation. The data show that, while UGI and dextran sulfate alone increase the likelihood that the target will be edited, as demonstrated by the increase in the percentage of reads which contain at least one C-to-T substitution, the combination of UGI and dextran sulfate yields a higher likelihood of editing than either alone. In many cases, the effect is more than additive.

TABLE 2

| | % of reads with any C-to-T substitution | | | | | | | |
|---|---|---|---|---|---|---|---|---|
| | Without SSB | | | | With SSB | | | |
| | EMX1-15 | HBB03 | HEKSite2 | RNF2 | EMX1-15 | HBB03 | HEKSite2 | RNF2 |
| RNP only | 14.94% ± | 13.55% ± | 10.89% ± | 5.64% ± | 11.96% ± | 7.33% ± | 10.26% ± | 4.91% ± |
| | 1.65% | 0.88% | 0.01% | 0.49% | 0.66% | 0.88% | 0.11% | 0.09% |
| RNP + UGI | 23.57% ± | 13.71% ± | 22.97% ± | 10.62% ± | 16.45% ± | 9.21% ± | 35.00% ± | 9.21% ± |
| | 1.66% | 0.86% | 2.60% | 0.93% | 0.59% | 0.46% | 2.26% | 0.51% |
| RNP + dextran | 32.79% ± | 57.13% ± | 16.13% ± | 33.13% ± | 49.22% ± | 45.94% ± | 16.51% ± | 37.48% ± |
| sulfate | 0.95% | 1.59% | 0.76% | 4.32% | 1.01% | 3.60% | 0.41% | 2.07% |
| RNB + UGI + | 62.25% ± | 53.44% ± | 35.13% ± | 56.89% ± | 71.16% ± | 53.97% ± | 50.37% ± | 67.09% ± |
| dextran sulfate | 6.39% | 7.59% | 4.32% | 14.26% | 4.79% | 6.78% | 2.32% | 8.80% |

In FIG. 4A-4H, the percentage of reads with a substitution at each cytosine in the protospacer is plotted, with C-to-T in grey, C-to-G in white, and C-to-A in black. (A) & (E) target EMX1-15; (B) & (F) target HEKSite2; (C) & (G) target HBB03 (D) & (H) target RNF2. Figs A-D contain data for the minimal CBE variant, which does not contain the SSB domain; Figs E-H contain data for the CBE variant with the N-terminal SSB domain. The data further confirm that the combination of UGI and dextran sulfate yields a higher rate of C-to-T conversion at positions within the editing window than either component alone. Further, the combination of UGI and dextran sulfate yields reduced rates of undesired C-to-G and C-to-A substitutions compared with dextran sulfate alone.

### Example 5

Addition of Dextran Sulfate to the Co-Transfection of Uracil Glycosylase Inhibitor Protein with CBE RNP Enhances the Reduction of C-A and C-G Substitution Outcomes by Uracil Glycosylase Inhibitor

Cytosine base editor (CBE) proteins were constructed and purified as in Example 4. Bacillus phage uracil glycosylase inhibitor (UGI) was purified as in Example 3. Synthetic single guide RNAs (sgRNAs) targeting four human genomic sites were purchased from MilliporeSigma. Spacer sequences of the sgRNAs are given in Table 1. Each experimental condition was tested in two technical replicates. Ribonucleoprotein (RNP) complexes and dextran sulfate solution were prepared as in Example 4. HEK293 cells were cultured and transfected as in Example 4. Genomic DNA was purified, genomic target sites were amplified, and libraries were prepared, sequenced, and analyzed as described in Example 1.

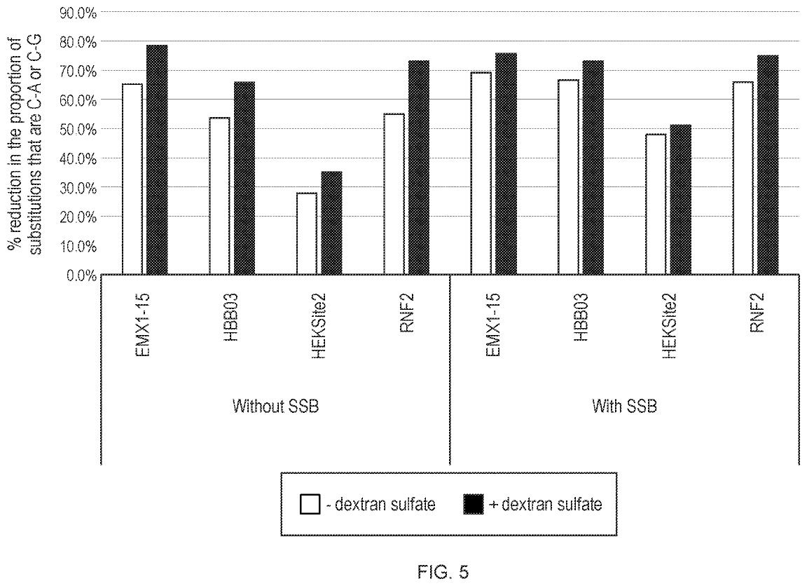

Results are presented in FIG. 5. For each CBE variant, RNPs were transfected alone and co-transfected with UGI protein, dextran sulfate, or both. The percent reduction in the proportion of substitutions that are C-to-A or C-to-G by UGI is plotted for transfections with (dark grey) and without (light grey) dextran sulfate. The data show that, when dextran sulfate is included in the transfection, the effect of the UGI on reducing C-to-A and C-to-G substitutions is enhanced. When 40 pmol of the CBE variant containing the N-terminal SSB fusion was used, the effect of UGI was enhanced by 5-15%. When 80 pmol of the CBE variant without the SSB fusion was used, the effect of UGI was enhanced by 20-35%.

### Example 6

Increasing the Length of sgRNA Shifts the Editing Window Farther from the PAM

Cytosine base editor (CBE) proteins were constructed and purified as in Example 4. Synthetic single guide RNAs (sgRNAs) with spacers measuring 20 nt, 21 nt, and 22 nt in length targeting three human genomic sites were purchased from MilliporeSigma. Spacer sequences of the sgRNAs are given in Table 3. Each experimental condition was tested in two technical replicates. Dextran sulfate solution was prepared as in Example 4.

TABLE 3
sgRNA Spacer Sequences

| | | SEQ |
|---|---|---|
| | Sequence (5′ - 3′) | ID NO. |
| EMX1-15 20 nt | GCTCCCATCACATCAACCGG | 12 |
| spacer | | |
| EMX1-15 21 nt | GGCTCCCATCACATCAACCGG | 24 |
| spacer | | |
| EMX1-15 22 nt | GGGCTCCCATCACATCAACCGG | 25 |
| spacer | | |
| HBB03 20 nt spacer | CTTGCCCCACAGGGCAGTAA | 13 |
| HBB03 21 nt spacer | CCTTGCCCCACAGGGCAGTAA | 26 |
| HBB03 22 nt spacer | ACCTTGCCCCACAGGGCAGTAA | 27 |
| RNF2 20 nt spacer | GTCATCTTAGTCATTACCTG | 15 |
| RNF2 21 nt spacer | AGTCATCTTAGTCATTACCTG | 28 |
| RNF2 22 nt spacer | CAGTCATCTTAGTCATTACCTG | 29 |

Ribonucleoprotein (RNP) complexes were prepared by incubating for 15 min at room temperature 40 pmol CBE protein and 120 pmol sgRNA in buffer (20 mM HEPES, 100 mM KCl, 0.5 mM DTT, 0.1 mM EDTA, pH 7.5) for a final volume of 10 μL. Transfections were performed in HEK293 cells as described in Example 4. Genomic DNA was purified, genomic target sites were amplified, and libraries were prepared, sequenced, and analyzed as described in Example 3. PCR primers are listed in Table 1.

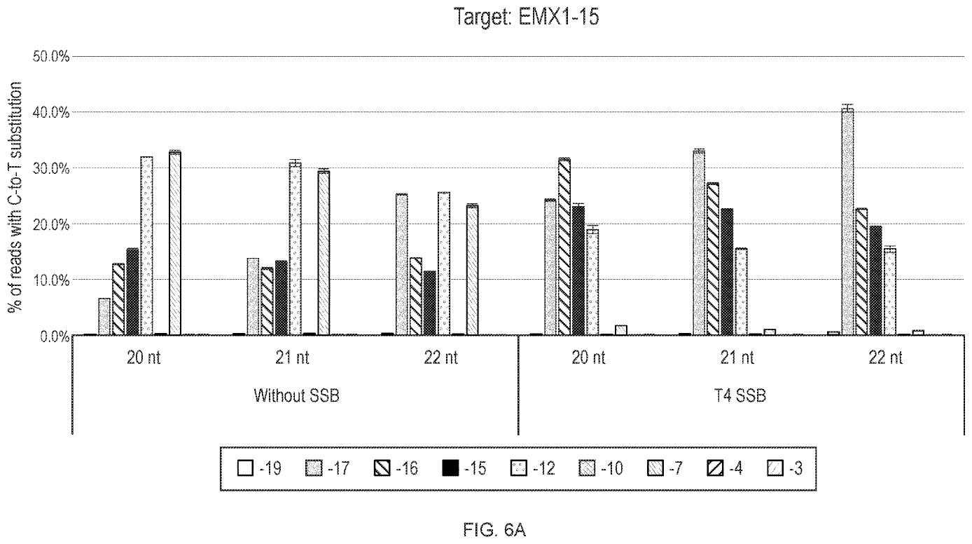

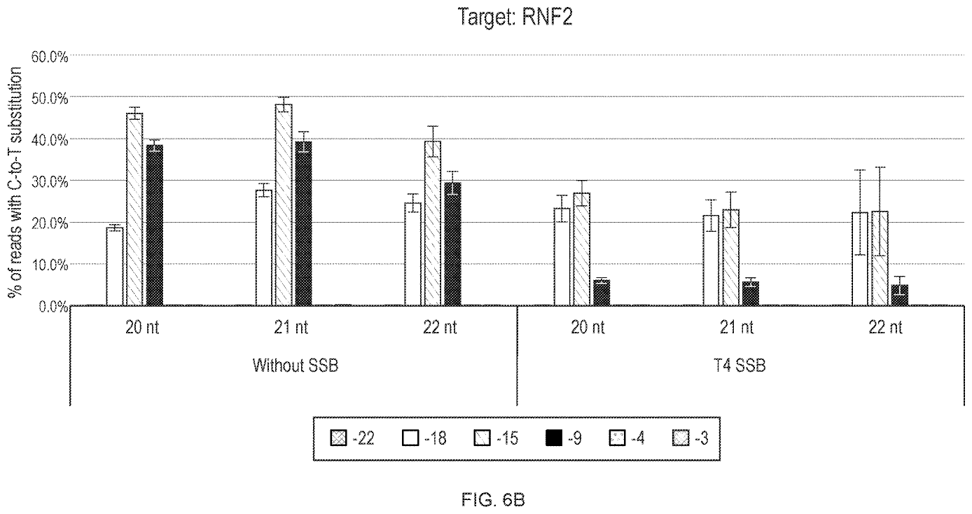

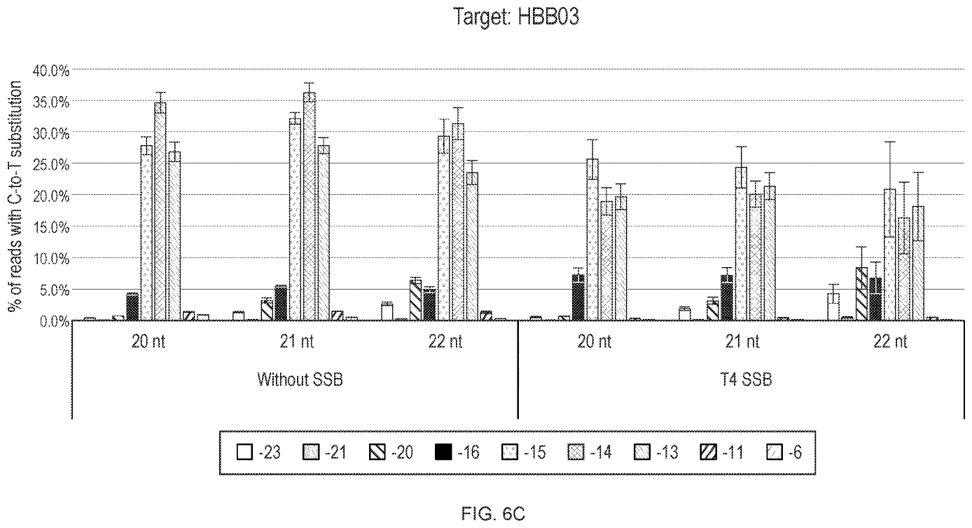

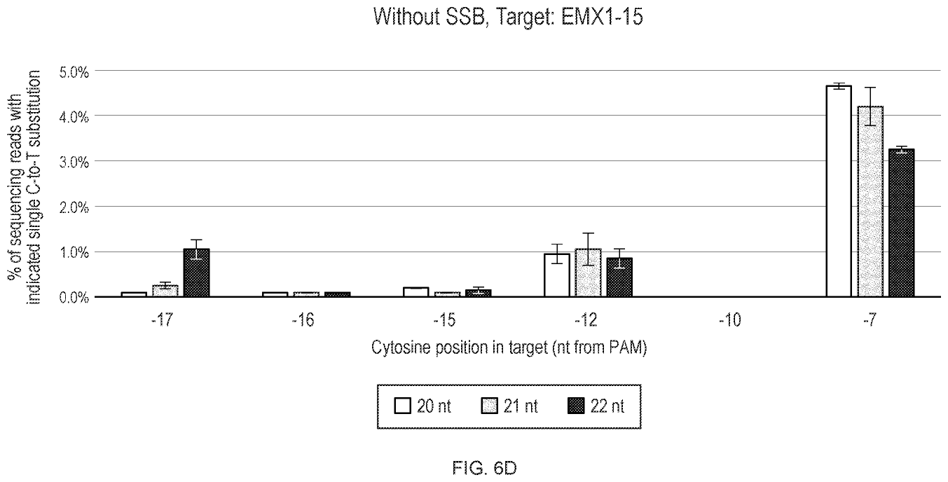

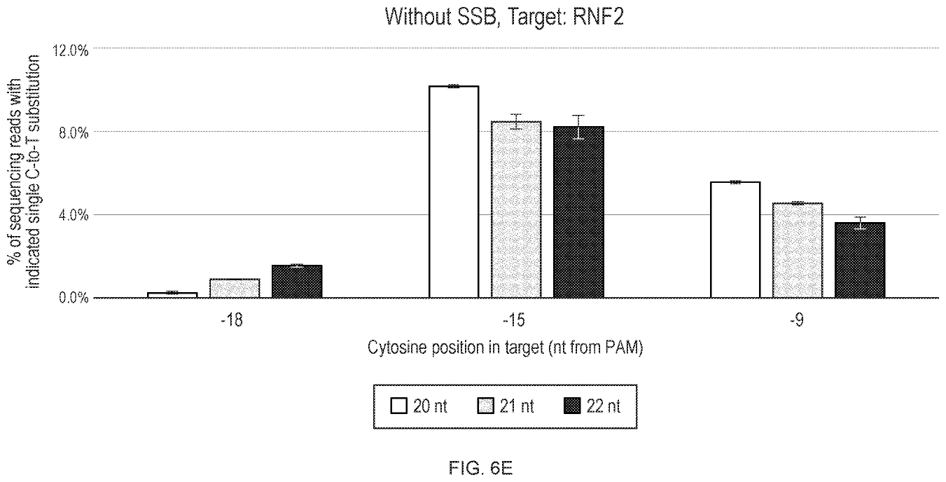

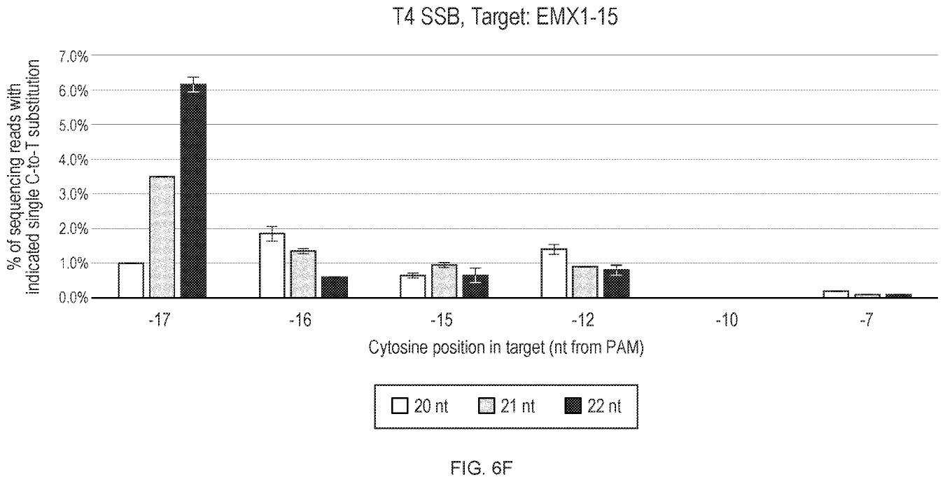

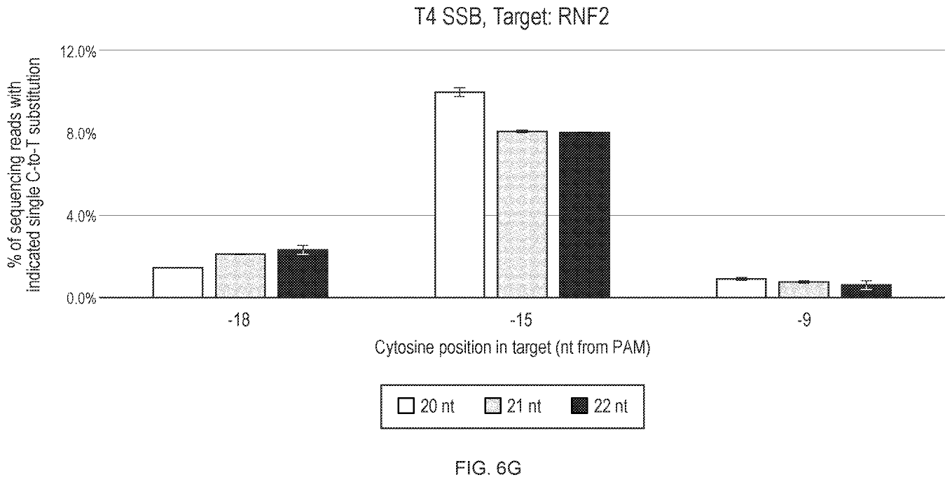

Results are presented in FIG. 6. In FIGS. 6A-6C, the percentage of reads with a C-to-T substitution at each cytosine in the protospacer is plotted. In these plots, protospacer cytosines are denoted as distance from the PAM (for example, position −3 is three nucleotides upstream of the PAM). Values are average±standard deviation for two replicates. The data shows that, as sgRNA length increases, editing increases at positions more distal to the PAM. In the case of target HBB03, this includes the cytosine residue at position −23, which is outside the sgRNA-targeting sequence of even the longest guide tested. 6A-6C show the percentage of reads with a C-to-T substitution for three targets and three spacer lengths: (A) target EMX1-15; (B) target RNF2; (C) target HBB03.

In FIGS. 6D-6G, the percentage of reads carrying single-residue editing alleles is plotted. Values are average±standard deviation for two replicates. This data shows that lengthening the guide results in an increase in the absolute rate of alleles with a single C-to-T edit at positions distal to the PAM and a concomitant decrease in the rate of alleles with a single C-to-T edit at PAM-proximal positions. When the target site contains multiple editable cytosines, and the desired edit is PAM-distal, lengthening the guide may facilitate obtaining the desired edit. 6D-6G show the percentage of reads carrying single-reside editing alleles: (D) target EMX1-15 without SSB; (E) target RNF2 without SSB; (F) target EMX1-15 with T4 SSB; (G) target RNF2 with T4 SSB.

### Example 7

In Vitro Transcribed T4 SSB-Containing Cytosine Base Editor and UGI mRNAs Improve Base Editing Efficiency and Precision

Plasmid vectors encoding the APOBEC3B derived cytosine base editor (SEQ ID NO: 10; designated here as A3B), the T4 phage SSB containing APOBEC3B derived cytosine base editor (SEQ ID NO: 11; designated here as T4 SSB-A3B), and the free UGI (SEQ ID NO: 3), as described in Examples 2 and 3, were each constructed with human codon optimization. Each gene was preceded by a T7 RNA Polymerase promoter for CleanCap (TriLink Biotechnologies, San Diego, CA) in vitro RNA transcription. Each plasmid was linearized by restriction digestion with Pmel (New England Biolabs, Ipswich, MA) and purified by two rounds of phenol/chloroform extraction. Linearized plasmid DNA was then used for mRNA production using a HiScribe T7 mRNA Kit (New England Biolabs, Ipswich, MA) and CleanCap Reagent AG (TirLink Bio). mRNA quality was verified on an Agilent 2100 Bioanalyzer using an RNA 6000 Nano kit (Agilent, Santa Clara, CA).

Human K562 cells harboring a Y93H (a T-to-C mutation in DNA) mutation in a GFP integrated at the human EMX1 locus were used for the experiment. The mutation abolished the GFP fluorophore formation and thus inactivated the protein fluorescent activity. Cells were retrieved from liquid nitrogen and grown in Iscove's Modified Dulbecco's Medium (SigmaAldrich, St. Louis, MO), supplemented with 10% FBS and 2 mM L-glutamine, at 37° C. and 5% CO₂ for a week. One day prior to transfection, cells were seeded at 0.25×10⁶ cells per mL and were at approximately 0.5×10⁶ cells per mL at the time of transfection. Cells were washed twice with Hank's Balanced Salt Solution and then resuspended in Nucleofector Solution V (Lonza; Bend, OR) at approximately 0.6×10⁶ cells per 100 μL. Transfection samples each contained 8 μg of a base editor mRNA and 200 pmol of sgRNA with the guide sequence of 5′-CUGAAGGUCACGUACAAGAG-3′ (SEQ ID NO: 41). A subset of the samples each also contained 4 μg of UGI mRNA. RNase-free water was used as negative control. The molar ratio of UGI mRNA to base editor mRNA was approximately 5:1. Nucleofection was performed by first mixing 100 μL of cells with transfection sample by gently pipetting up and down without introducing air bubbles before transferring into a cuvette for immediate electroporation on an Amaxa instrument (Lonza) using Program T-016. Cells were immediately transferred to a 6-well plate containing 2 mL pre-warmed medium per well after nucleofection and then grown at 37° C. and 5% CO₂.

Flow cytometry analysis was performed on a MACSQuant Analyzer (Miltenyi Biotec, San Diego, CA) five days after transfection and data were analyzed using a FlowJo program (BD Biosciences, Franklin Lake, NJ). Genomic DNA was harvested from transfected cells five days after transfection using QuickExtract Solution (Lucigen, Middleton, WI). Targeted genomic region was PCR amplified with a pair of NGS primers using JumpStart™ Taq ReadyMix™ for Quantitative PCR Kit (MilliporeSigma, Burlington, MA) with the following cycling condition: 98° C./2 m; 98° C./15 s, 62° C./30 s, and 72° C./45 s for 34 cycles; 72° C./5 m. The NGS primers were: 5′-TCGTCGGCAGCGTCAGATGTGTATAAGAGACAGNNNNNNCCTGAAGTTCAT CTGCACCACC-3′ (SEQ ID NO: 42) (forward) and 5′-GTCTCGTGGGCTCGGAGATGTGTATAAGAGACAGNNNNNNCACGTTGTGGC TGTTGTAGTTGTA-3′ (SEQ ID NO: 43) (reverse). Primary PCR products were then reamplified with Illumina index primers using JumpStart™ Taq ReadyMix™ for Quantitative PCR Kit (MilliporeSigma, Burlington, MA) with the following cycling condition: 95° C./3 m; 95° C./30 s, 55° C./30 s, and 72° C./30 s for 8 cycles; 72° C./5 m. Indexed PCR products were purified with Select-α-Size DNA Clean & Concentrator kit (Zymo, Irvine, CA) and quantified by PicoGreen (ThermoFisher, Waltham, MA). PCR products were then normalized and pooled to make NGS libraries. NGS was performed using an Illumina MiSeq instrument and a 2×300 bp kit (San Diego, CA). FASTQ files for each sample were analyzed using a custom analysis program.

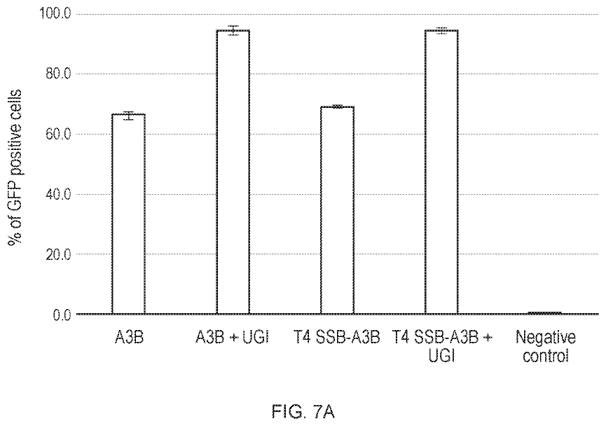

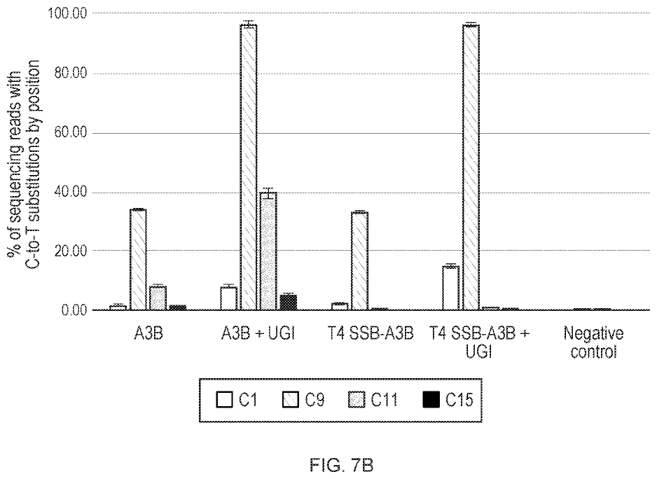

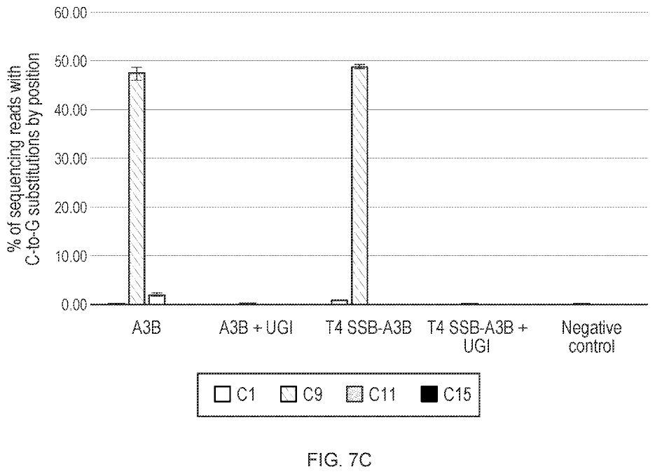

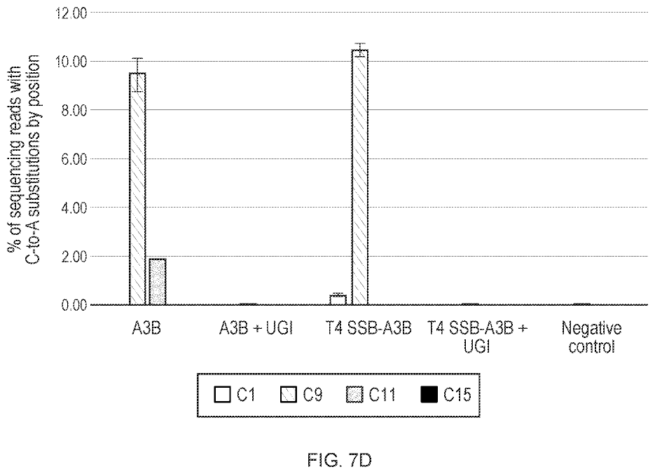

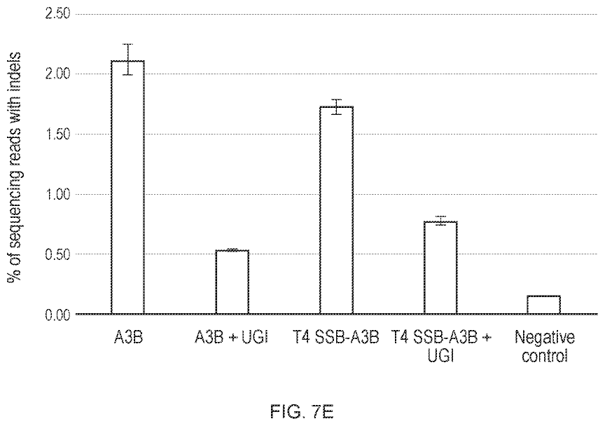

Results are presented in FIGS. 7A-7E. FIG. 7A shows the percentages of cells that became GFP positive after transfection with the two cytosine base editor mRNAs (A3B and T4 SSB-A3B) with or without the presence of the UGI mRNA. The results demonstrate that adding the UGI mRNA into the transfection increased the number of GFP positive cells by about 50%. FIGS. 7B-7D show the percentages of sequencing reads with C-to-T (FIG. 7B), C-to-G (FIG. 7C), or C-to-A (FIG. 7D) substitutions by positions from NGS analysis of transfected cells. FIG. 7E shows the percentages of sequencing reads with indels from NGS analysis of transfected cells. These results demonstrate that adding the UGI mRNA into the transfection increased C-to-T substitutions and at the same time reduced C-to-G or C-to-A substitutions and indel formation. These results further demonstrate that the T4 SSB containing base editor mRNA reduced the off-target effects on two of the three unintended C nucleotides (C1, C11, and C15) within the protospacer compared to the base editor mRNA without the SSB domain, while these two base editor mRNAs had a similar C-to-T conversion efficiency on the intended C9 nucleotide.

### Example 8

UGI Used with a Nuclear Location Sequence (NLS)

Cytosine base editor (CBE) proteins were constructed by fusing the C-terminal domain of human APOBEC3B to the amino-terminus of an SpCas9 nickase protein via a rigid peptide linker and two uracil glycosylase inhibitor (UGI) domains to the C-terminus (SEQ ID NO: 12). A recombinant UGI was constructed by fusing a Bacillus phage UGI, a c-MYC nuclear localization signal (NLS), and a SV40 Large T antigen NLS (SEQ ID NO: 3). Recombinant UGI not containing NLS sequence was purchased from NEB (Cat No M0281). All proteins were expressed and purified from E. coli BL21AI by autoinduction and nickel column chromatography and were stored at −80° C. before use in a buffer containing 10% glycerol, 300 mM KCl, 20 mM HEPES (pH 7.5), and 1 mM DTT. Synthetic single guide RNAs (sgRNAs) targeting EMX1-15 and HEKSite2 were purchased from MilliporeSigma. Spacer sequences of the sgRNAs are given in Table 1. Each experimental condition was tested in two technical replicates.

Ribonucleoprotein (RNP) complexes were prepared by incubating for 15 min at room temperature: 15 μg of CBE protein, 200 pmol sgRNA, and 158 to 1185 pmol UGI in buffer (20 mM HEPES, 100 mM KCl, 0.5 mM DTT, 0.1 mM EDTA, pH 7.5) for a final volume of 10 μL. The molar ratio of CBE to sgRNA was approximately 1:3. RNPs were kept on ice until transfection. HEK293 cells were obtained from ATCC and grown at 37° C. and 5% CO₂ in DMEM supplemented with 10% FBS, 2 mM L-glutamine, 1 mM sodium pyruvate, and 0.1 mM non-essential amino acids. Cells were seeded at 1.67×10⁴ cells/cm² of tissue culture surface area two days before transfection. At the time of transfection, cells were trypsinized to obtain a single-cell suspension, washed twice with Hank's Balanced Salt Solution and resuspended in Nucleofector Solution V (Lonza) at 2.5×10⁵ cells per 100 μL. Nucleofection was performed by mixing 100 μL of prepared cell suspension with 10 μL of complexed CBE RNP by pipetting up and down six times before transferring to a cuvette for electroporation using program Q-001 on a Nucleofector 2b machine (Lonza). Nucleofected cells were immediately transferred to 6-well plates containing 2 mL pre-warmed media per well and grown for 3 days before harvest.

Genomic DNA was harvested by trypsinizing transfected cells and resuspending in 50 μL QuickExtract reagent (Lucigen). Suspensions were incubated at 60° C. for 15m and 95° C. for 15m. Genomic regions targeted by the CBE were amplified by PCR using JumpStart Taq ReadyMix (MilliporeSigma) and the following cycling conditions: 94° C./2 m; 25 cycles of 94° C./30 s, 62° C./30s, 72° C./45 s; 72° C./5 m. Primers are listed in Table 1. PCR products underwent a second round of amplification using Illumina index primers and JumpStart Taq ReadyMix and the following conditions: 95° C./3 m; 9 cycles of 95° C./30 s, 55° C./30 s, 72° C./30 s; 72° C.° C./5 m. Indexed PCR products were purified by Select-α-Size DNA Clean & Concentrator MagBeads (Zymo, Irvine, CA), using 1.2× beads by volume, quantified by PicoGreen (ThermoFisher, Waltham, MA), and pooled according to DNA content. Pools were diluted to 4 nM. Sequencing was performed on an Illumina MiSeq instrument using a 300-cycle kit to obtain single-end reads. FASTQ files for each sample were analyzed using the CRIS.py software package (Connelly & Pruett-Miller, Sci. Rep., 2019).

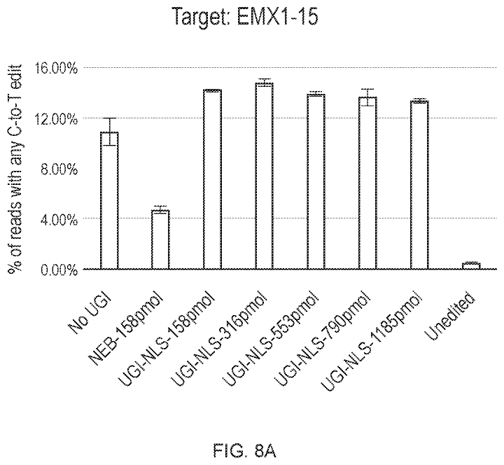

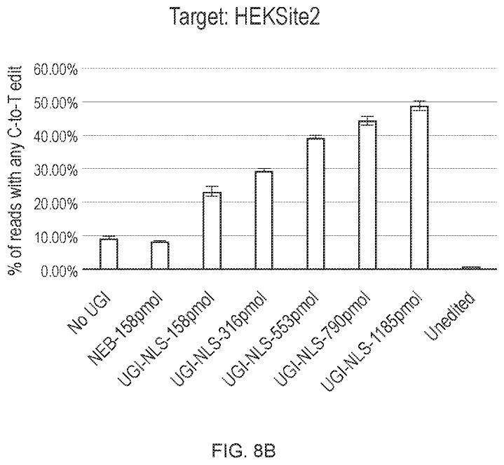

Results are presented in FIGS. 8A & 8B. For each target: (A) EMX1-1; and (B) HEKSite2; the percentage of reads with any C-to-T edit is plotted for each UGI condition as described above and as given in the X-axis of the graphs. Values are average±standard deviation for two replicates. For both target sites, co-delivery of UGI protein with an NLS results in an increased C-to-T editing rate. In contrast, the NEB UGI, which does not have an NLS, exhibits editing at a rate similar to or lower than the control samples without standalone UGI protein.

We claim:

1. A composition suitable for modifying a cytosine residue in a DNA sequence, comprising: a single stranded DNA binding domain (SSB), a deaminase, a catalytically modified Cas protein, a guide RNA and, optionally, one or more free and/or fused uracil glycosylase inhibitors (UGI) to form a base editing RNP complex optionally with UGI.

2. The composition of claim 1, wherein the SSB is linked directly to said deaminase.

3. The composition of claim 1, wherein the SSB is linked indirectly to said deaminase.

4. The composition of claim 1, wherein said SSB is not covalently linked to any of the deaminase, the catalytically modified Cas protein, the guide RNA or the optional UGI.

5. The composition of claim 1, wherein said deaminase is covalently linked to the catalytically modified Cas protein.

6. The composition of claim 1, wherein said one or more UGI are not covalently linked to any of the deaminase, the catalytically modified Cas protein or the guide RNA.

7. The composition of claim 1, wherein the one or more SSB are from viruses.

8. The composition of claim 1, wherein the one or more SSB are from prokaryotes.

9. The composition of claim 1, wherein the one or more SSB are from eukaryotes.

10. The composition of claim 1, wherein said deaminase is a cytosine deaminase.

11. The composition of claim 1, wherein said an adenosine deaminase.

12. The composition of claim 1, wherein the catalytically modified Cas protein contains a catalytically inactive RuvC nuclease domain.

13. The composition of claim 1, wherein the catalytically modified Cas protein contains a catalytically inactive RuvC nuclease domain and a catalytically inactive HNH nuclease domain.

14. The composition of claim 1, wherein the catalytically modified Cas protein is a Cas9.

15. The composition of claim 1, wherein the catalytically modified Cas protein is a Cas12.

16. At least one nucleic acid encoding one or more of the SSB, the deaminase, the catalytically modified Cas protein, the guide RNA and, optionally, the one or more free and/or fused UGI of the composition of claim 1.

17. At least one expression vector comprising a nucleic acid encoding at least one or more of the SSB, the deaminase, the catalytically modified Cas protein, the guide RNA and, optionally, the one or more free and/or fused UGI of the composition of claim 1.

18. The composition of claim 1 further comprising a Nuclear Location Sequence (NLS).

19. A composition suitable for modifying a cytosine residue in a DNA sequence, comprising: a deaminase, a catalytically modified Cas protein, a guide RNA and one or more free UGI to form a base editing RNP complex with free UGI.

20. The composition of claim 19, wherein said deaminase is covalently linked to the catalytically modified Cas protein.

21. The composition of claim 19, wherein said one or more UGI are not covalently linked to any of the deaminase, the catalytically modified Cas protein or the guide RNA.

22. The composition of claim 19, wherein said deaminase a cytosine deaminase.

23. The composition of claim 19, wherein said deaminase is an adenosine deaminase

24. The composition of claim 19, wherein the catalytically modified Cas protein contains a catalytically inactive RuvC nuclease domain.

25. The composition of claim 19, wherein the catalytically modified Cas protein contains a catalytically inactive RuvC nuclease domain and a catalytically inactive HNH nuclease domain.

26. The composition of claim 19, wherein the catalytically modified Cas protein is a Cas9.

27. The composition of claim 19, wherein the catalytically modified Cas protein is a Cas12.

28. The composition of claim 19, wherein said deaminase, said catalytically modified Cas protein, said guide RNA are encoded in one or more nucleic acids and, wherein said UGI is a peptide or encoded in said one or more nucleic acid.

29. One or more expression vectors comprising one or more of the nucleic acids of claim 28.

30. The composition of claim 19 further comprising a Nuclear Location Sequence (NLS).

31.-65. (canceled)


SEQUENCE LISTING (44 sequences)

SEQ ID NO: 1 - protein, 235 aa, synthetic construct
KGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKA

SEQ ID NO: 2 - protein, 211 aa, synthetic construct
MAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPR

SEQ ID NO: 3 - protein, 115 aa, synthetic construct
MTNLSDIIEKETGKQLVIQESILMLPEEVEEVIGNKPESDILVHTAYDESTDENVMLLTSDAPEYKPWALVIQDSNGENKIKMLSGGSGGSGGSPAAKRVKLDGGGGSPKKKRKV

SEQ ID NO: 4 - protein, 1841 aa, synthetic construct
GPGSMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNASSGGSSGGSSGSETPGTSESATPESSGGSSGGSPAAKRVKLDGGGGSPKKKRKVTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSRSPKKKRKVGGGGSPKKKRKV

SEQ ID NO: 5 - protein, 1834 aa, synthetic construct
GPGSEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQASSGGSSGGSSGSETPGTSESATPESSGGSSGGSPAAKRVKLDGGGGSPKKKRKVTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSRSPKKKRKVGGGGSPKKKRKV

SEQ ID NO: 6 - protein, 2046 aa, synthetic construct
GPGSKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKAGGGGSVDMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNASSGSETPGTSESATPESTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSRSPKKKRKVGGGGSPKKKRKV

SEQ ID NO: 7 - protein, 2022 aa, synthetic construct
GPGSMAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRGGGGSVDMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNASSGSETPGTSESATPESTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSRSPKKKRKVGGGGSPKKKRKV

SEQ ID NO: 8 - protein, 1847 aa, synthetic construct
GPGSKGFSSEDKGEWKLKLDNAGNGQAVIRFLPSKNDEQAPFAILVNHGFKKNGKWYIETCSSTHGDYDSCPVCQYISKNDLYNTDNKEYSLVKRKTSYWANILVVKDPAAPENEGKVFKYRFGKKIWDKINAMIAVDVEMGETPVDVTCPWEGANFVLKVKQVSGFSNYDESKFLNQSAIPNIDDESFQKELFEQMVDLSEMTSKDKFKSFEELNTKFGQVMGTAVMGGAAATAAKKAGGGGSVDEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQASSGSETPGTSESATPESTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVGGGGSPKKKRKV

SEQ ID NO: 9 - protein, 2015 aa, synthetic construct
GPGSMAKKIFTSALGTAEPYAYIAKPDYGNEERGFGNPRGVYKVDLTIPNKDPRCQRMVDEIVKCHEEAYAAAVEEYEANPPAVARGKKPLKPYEGDMPFFDNGDGTTTFKFKCYASFQDKKTKETKHINLVVVDSKGKKMEDVPIIGGGSKLKVKYSLVPYKWNTAVGASVKLQLESVMLVELATFGGGEDDWADEVEENGYVASGSAKASKPRGGGGSVDEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQASSGSETPGTSESATPESTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSRSPKKKRKVGGGGSPKKKRKV

SEQ ID NO: 10 - protein, 1642 aa, synthetic construct
GPGSEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQASSGGSSGGSSGSETPGTSESATPESSGGSSGGSPAAKRVKLDGGGGSPKKKRKVTGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEFPKKKRKVGGGGSPKKKRKV

SEQ ID NO: 11 - protein, 1847 aa, synthetic construct; identical to SEQ ID NO: 8

SEQ ID NO: 12 - DNA, 20 bp, synthetic construct
  source 1..20: mol_type = other DNA
gctcccatcacatcaaccgg

SEQ ID NO: 13 - DNA, 20 bp, synthetic construct
  source 1..20: mol_type = other DNA
cttgccccacagggcagtaa

SEQ ID NO: 14 - DNA, 20 bp, synthetic construct
  source 1..20: mol_type = other DNA
gaacacaaagcatagactgc

SEQ ID NO: 15 - DNA, 20 bp, synthetic construct
  source 1..20: mol_type = other DNA
gtcatcttagtcattacctg

SEQ ID NO: 16 - DNA, 61 bp, synthetic construct
  source 1..61: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
tcgtcggcagcgtcagatgtgtataagagacagnnnnnncctgagtttctcatctgtgccc

SEQ ID NO: 17 - DNA, 60 bp, synthetic construct
  source 1..60: mol_type = other DNA
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
gtctcgtgggctcggagatgtgtataagagacagnnnnnntgactccaggcctccccaaa

SEQ ID NO: 18 - DNA, 59 bp, synthetic construct
  source 1..59: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
tcgtcggcagcgtcagatgtgtataagagacagnnnnnngccaatctactcccaggagc

SEQ ID NO: 19 - DNA, 62 bp, synthetic construct
  source 1..62: mol_type = other DNA
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
gtctcgtgggctcggagatgtgtataagagacagnnnnnnaggcagagagagtcagtgccta

SEQ ID NO: 20 - DNA, 63 bp, synthetic construct
  source 1..63: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
tcgtcggcagcgtcagatgtgtataagagacagnnnnnntgaacttcccaagtgagaagccag

SEQ ID NO: 21 - DNA, 60 bp, synthetic construct
  source 1..60: mol_type = other DNA
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
gtctcgtgggctcggagatgtgtataagagacagnnnnnntggcaggacgtctgcccaat

SEQ ID NO: 22 - DNA, 69 bp, synthetic construct
  source 1..69: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
tcgtcggcagcgtcagatgtgtataagagacagnnnnnntgttagccaacatacagaagtcaggaatgc

SEQ ID NO: 23 - DNA, 64 bp, synthetic construct
  source 1..64: mol_type = other DNA
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
gtctcgtgggctcggagatgtgtataagagacagnnnnnnttcagaccatagcacttcccttcc

SEQ ID NO: 24 - DNA, 21 bp, synthetic construct
  source 1..21: mol_type = other DNA
ggctcccatcacatcaaccgg

SEQ ID NO: 25 - DNA, 22 bp, synthetic construct
  source 1..22: mol_type = other DNA
gggctcccatcacatcaaccgg

SEQ ID NO: 26 - DNA, 21 bp, synthetic construct
  source 1..21: mol_type = other DNA
ccttgccccacagggcagtaa

SEQ ID NO: 27 - DNA, 22 bp, synthetic construct
  source 1..22: mol_type = other DNA
accttgccccacagggcagtaa

SEQ ID NO: 28 - DNA, 21 bp, synthetic construct
  source 1..21: mol_type = other DNA
agtcatcttagtcattacctg

SEQ ID NO: 29 - DNA, 22 bp, synthetic construct
  source 1..22: mol_type = other DNA
cagtcatcttagtcattacctg

SEQ ID NO: 30 - protein, 7 aa, synthetic construct
PKKKRKV

SEQ ID NO: 31 - protein, 7 aa, synthetic construct
PKKKRRV

SEQ ID NO: 32 - protein, 16 aa, synthetic construct
KRPAATKKAGQAKKKK

SEQ ID NO: 33 - protein, 20 aa, Human immunodeficiency virus 1
GRKKRRQRRRPPQPKKKRKV

SEQ ID NO: 34 - protein, 19 aa, Hepacivirus B
PLSSIFSRIGDPPKKKRKV

SEQ ID NO: 35 - protein, 24 aa, synthetic construct
GALFLGWLGAAGSTMGAPKKKRKV

SEQ ID NO: 36 - protein, 27 aa, synthetic construct
GALFLGFLGAAGSTMGAWSQPKKKRKV

SEQ ID NO: 37 - protein, 21 aa, synthetic construct
KETWWETWWTEWSQPKKKRKV

SEQ ID NO: 38-40 - skipped (3 entries)

SEQ ID NO: 41 - RNA, 20 nt, synthetic construct
  source 1..20: mol_type = other RNA
ctgaaggtcacgtacaagag

SEQ ID NO: 42 - DNA, 61 bp, synthetic construct
  source 1..61: mol_type = other DNA
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
tcgtcggcagcgtcagatgtgtataagagacagnnnnnncctgaagttcatctgcaccacc

SEQ ID NO: 43 - DNA, 64 bp, synthetic construct
  source 1..64: mol_type = other DNA
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
gtctcgtgggctcggagatgtgtataagagacagnnnnnncacgttgtggctgttgtagttgta

SEQ ID NO: 44 - protein, 6 aa, synthetic construct
HHHHHH